(19) United States
(12) Patent Application Publication
(10) Pub. No.: US 2023/0407324 A1
(43) Pub. Date: Dec. 21, 2023

(54) DOUBLED HAPLOID INDUCER

(71) Applicant: PIONEER HI-BRED INTERNATIONAL, INC., JOHNSTON, IA (US)

(72) Inventors: JON AARON TUCKER REINDERS, CLIVE, IA (US); MARK E. WILLIAMS, JOHNSTON, IA (US); HUAXUN YE, JOHNSTON, IA (US)

(73) Assignee: PIONEER HI-BRED INTERNATIONAL, INC., JOHNSTON, IA (US)

(21) Appl. No.: 18/249,891

(22) PCT Filed: Oct. 20, 2021
(86) PCT No.: PCT/US21/71946

§ 371 Date: Apr. 20, 2023

Related U.S. Application Data
(60) Provisional application No. 63/094,830, filed on Oct. 21, 2020.

(51) Int. Cl. C12N 15/82; A01H 1/08

(52) U.S. Cl. CPC C12N 15/8261; C12N 15/8201; A01H 1/08

(57) ABSTRACT

Methods of creating doubled haploid inducers are provided. These doubled haploid inducers are used in the production of maternal doubled haploids.

### CROSS-REFERENCE TO RELATED APPLICATIONS

This application claims priority to U.S. Provisional Application No. 63/094,830, filed Oct. 21, 2020, which is hereby incorporated herein by reference in its entirety.

### FIELD

The present disclosure relates to the field of plant molecular biology and plant breeding.

### REFERENCE TO SEQUENCE LISTING SUBMITTED ELECTRONICALLY

The official copy of the sequence listing is submitted electronically via EFS-Web as an ASCII formatted sequence listing with a file named 20211010_8536-WO-PCT_ST25 created on Oct. 10, 2021 and having a size of 1,131,526 bytes and is filed concurrently with the specification. The sequence listing contained in this ASCII formatted document is part of the specification and is herein incorporated by reference in its entirety.

### BACKGROUND OF THE DISCLOSURE

Plant breeding programs identify new cultivars by screening numerous plants to identify individuals with desirable characteristics. Large numbers of progeny from crosses are typically grown and evaluated, ideally across multiple years and environments, to select the plants with the most desirable characteristics.

Typical breeding methods cross two parental plants and the filial 1 hybrid (F₁ hybrid), is the first filial generation. Hybrid vigor in a commercial F₁ hybrid is observed when two parental strains, (typically inbreds), from different heterotic groups are intercrossed. Hybrid vigor, the improved or increased function of any biological quality resulting from combining the genetic contributions of its parents, is important to commercial maize seed production. Commercial hybrid performance improvements require continued development of new inbred parental lines.

Maize inbred line development methods may use maternal (gynogenic) doubled haploid production, in which maternal haploid embryos are selected following the fertilization of the ear of a plant resultant from a first-generation cross that has been fertilized with pollen from a so-called “haploid inducer” line. Pollination of a female flower with pollen of a haploid inducer line (pollen donor) results in elevated levels of ovules that contain only the haploid (1n) maternal genome, as opposed to inheriting a copy of both the maternal and paternal genome, thus, creating maternal haploid embryos. Ovules within the female flower are the products of meiosis and each maternal ovule is a unique meiotically recombined haploid genome, thereby allowing immature maternal haploid embryos to be isolated and treated using in vitro tissue culture methods that include chromosome doubling treatments to rapidly enable generating maternal doubled haploid (2n) recombinant populations comprising paired maternal chromosomes. Many of the maize maternal haploid embryos generated by fertilizing a target plant with pollen from a maize haploid inducer line fail to regenerate into a fertile, doubled haploid plant and few, if any, in vitro tissue culture and plantlet regeneration methods propagate multiple, fertile plants from one haploid embryo. In addition, maternal haploid embryos produce a mixoploid plant having a mixture of haploid, diploid and/or polyploid cells that produce low amounts of seed. This mixoploidy hinders initial phenotypic evaluations of these plants. Further, the isolation of maternal haploid embryos is labor intensive and time-consuming. Some chemical doubling agents are harmful to mammalian cells at certain concentrations. Thus, there is a need for improving methods of producing doubled haploid plants from maternal gamete doubled haploids in maize.

Plant breeders would thus also benefit from methods of developing a population of recombinant inbred lines that do not require extensive pollination control methods or the prolonged time required for propagating self-fertilized lines into isogenic states.

### SUMMARY OF THE DISCLOSURE

The present disclosure provides a method of producing a doubled haploid plant, comprising a) providing a first plant, wherein the first plant comprises at least one introduced genetic chromosome doubling agent; b) crossing the first plant with a second plant, wherein the at least one introduced genetic chromosome doubling agent of the first plant induces chromosome doubling of a fertilized egg cell of the second plant in the absence of an exogenously applied chemical or biochemical chromosome doubling agent; c) obtaining a diploidized embryo comprising a pair of chromosomes inherited from the second plant and not comprising the introduced genetic chromosome doubling agent; and d) regenerating a diploid plant from the diploidized embryo. In an aspect, the first plant is a haploid inducer. In an aspect, the haploid inducer comprises a loss-of-function mutation in a patatin-like phospholipase A2α gene. In an aspect, the loss-of-function mutation in the patatin-like phospholipase A2α gene is the MATRILINEAL (MATL) gene. In an aspect, the first plant expresses a marker gene. In an aspect, the marker gene is selected from a selectable marker, a reporter gene, a visible endogenous morphological marker, and combinations thereof. In an aspect, the selectable marker is selected from the group consisting of GUS, PMI, PAT, and combinations thereof. In an aspect, the reporter gene is selected from the group consisting of GFP, RFP, CFP, and combinations thereof. In an aspect, the visible endogenous morphological marker is selected from the group consisting of B1, R-nj, R1-scm, anthocyanin pigments, and combinations thereof. In an aspect, the genetic chromosome doubling agent is a genetic chromosome doubling polypeptide. In an aspect, the genetic chromosome doubling polypeptide is selected from the group consisting of a) a polypeptide allowing replication of a genome without cell division; b) a polypeptide destabilizing tubulin polymerization; c) a polypeptide altering cell cycle regulation; and d) combinations of the foregoing. In an aspect, the genetic chromosome doubling polypeptide allowing replication of the genome without cell division is selected from the group consisting of a) an amino acid sequence having at least 70% (e.g., at least 80%, 90%, 95%, or 98%) identical to SEQ ID NO: 24; b) an amino acid sequence having at least 70% (e.g., at least 80%, 90%, 95%, or 98%) identical to SEQ ID NO: 25; c) an amino acid sequence having at least 70% (e.g., at least 80%, 90%, 95%, or 98%) identical to SEQ ID NO: 26; or d) an amino acid sequence having at least 70% (e.g., at least 80%, 90%, 95%, or 98%) identical to SEQ ID NO: 2. In an aspect, the genetic chromosome doubling polypeptide destabilizing tubulin polymerization is selected from the group consisting of a) an amino acid sequence having at least 70% (e.g., at least 80%, 90%, 95%, or 98%) identical to SEQ ID NO: 3; orb) an amino acid sequence having at least 70% (e.g., at least 80%, 90%, 95%, or 98%) identical to SEQ ID NO: 28. In an aspect, the genetic chromosome doubling polypeptide altering cell cycle regulation is selected from the group consisting of a) an amino acid sequence having at least 70% (e.g., at least 80%, 90%, 95%, or 98%) identical to SEQ ID NO: 23; b) an amino acid sequence having at least 70% (e.g., at least 80%, 90%, 95%, or 98%) identical to SEQ ID NO: 86; c) an amino acid sequence having at least 70% (e.g., at least 80%, 90%, 95%, or 98%) identical to SEQ ID NO: 87; d) an amino acid sequence having at least 70% (e.g., at least 80%, 90%, 95%, or 98%) identical to SEQ ID NO: 88; e) an amino acid sequence having at least 70% (e.g., at least 80%, 90%, 95%, or 98%) identical to SEQ ID NO: 89; f) an amino acid sequence having at least 70% (e.g., at least 80%, 90%, 95%, or 98%) identical to SEQ ID NO: 90; g) an amino acid sequence having at least 70% (e.g., at least 80%, 90%, 95%, or 98%) identical to SEQ ID NO: 91; h) an amino acid sequence having at least 70% (e.g., at least 80%, 90%, 95%, or 98%) identical to SEQ ID NO: 92; i) an amino acid sequence having at least 70% (e.g., at least 80%, 90%, 95%, or 98%) identical to SEQ ID NO: 93; j) an amino acid sequence having at least 70% (e.g., at least 80%, 90%, 95%, or 98%) identical to SEQ ID NO: 94; k) an amino acid sequence having at least 70% (e.g., at least 80%, 90%, 95%, or 98%) identical to SEQ ID NO: 95; l) an amino acid sequence having at least 70% (e.g., at least 80%, 90%, 95%, or 98%) identical to SEQ ID NO: 96; m) an amino acid sequence having at least 70% (e.g., at least 80%, 90%, 95%, or 98%) identical to SEQ ID NO: 97; n) an amino acid sequence having at least 70% (e.g., at least 80%, 90%, 95%, or 98%) identical to SEQ ID NO: 98; o) an amino acid sequence having at least 70% (e.g., at least 80%, 90%, 95%, or 98%) identical to SEQ ID NO: 99; p) an amino acid sequence having at least 70% (e.g., at least 80%, 90%, 95%, or 98%) identical to SEQ ID NO: 100; q) an amino acid sequence having at least 70% (e.g., at least 80%, 90%, 95%, or 98%) identical to SEQ ID NO: 101; r) an amino acid sequence having at least 70% (e.g., at least 80%, 90%, 95%, or 98%) identical to SEQ ID NO: 102; or s) an amino acid sequence having at least 70% (e.g., at least 80%, 90%, 95%, or 98%) identical to SEQ ID NO: 103. In an aspect, the first plant is a haploid inducer of a different plant species than the second plant. In an aspect, the first plant is used in a wide hybridization. In an aspect, the second plant species is corn, soybean, wheat, brassica, cotton or sorghum. In an aspect, the second plant species is a non-hybrid crop species.

The present disclosure provides a method of producing a doubled haploid inducer (DHI) plant, the method comprising a) providing a transformable cell or tissue from a non-transgenic haploid inducer plant; b) transforming the cell or tissue with a heterologous DNA construct comprising a genetic chromosome doubling agent; and c) obtaining a double haploid inducer (DHI) line comprising the introduced genetic chromosome doubling agent. In an aspect, the non-transgenic haploid inducer comprises a loss-of-function mutation in a patatin-like phospholipase A2α gene. In an aspect, the loss-of-function mutation in the patatin-like phospholipase A2α gene is the MATRILINEAL (MATL) gene. In an aspect, the genetic chromosome doubling agent is selected from the group consisting of a) a polypeptide allowing replication of a genome without cell division; b) a polypeptide destabilizing tubulin polymerization; c) a polypeptide altering cell cycle regulation; and d) combinations of the foregoing. In an aspect, the genetic chromosome doubling polypeptide allowing replication of the genome without cell division is selected from the group consisting of a) an amino acid sequence having at least 70% (e.g., at least 80%, 90%, 95%, or 98%) identical to SEQ ID NO: 24; b) an amino acid sequence having at least 70% (e.g., at least 80%, 90%, 95%, or 98%) identical to SEQ ID NO: 25; c) an amino acid sequence having at least 70% (e.g., at least 80%, 90%, 95%, or 98%) identical to SEQ ID NO: 26; or d) an amino acid sequence having at least 70% (e.g., at least 80%, 90%, 95%, or 98%) identical to SEQ ID NO: 2. In an aspect, the genetic chromosome doubling polypeptide destabilizing tubulin polymerization is selected from the group consisting of a) an amino acid sequence having at least 70% (e.g., at least 80%, 90%, 95%, or 98%) identical to SEQ ID NO: 3; orb) an amino acid sequence having at least 70% (e.g., at least 80%, 90%, 95%, or 98%) identical to SEQ ID NO: 28. In an aspect, the genetic chromosome doubling polypeptide altering cell cycle regulation is selected from the group consisting of a) an amino acid sequence having at least 70% (e.g., at least 80%, 90%, 95%, or 98%) identical to SEQ ID NO: 23; b) an amino acid sequence having at least 70% (e.g., at least 80%, 90%, 95%, or 98%) identical to SEQ ID NO: 86; c) an amino acid sequence having at least 70% (e.g., at least 80%, 90%, 95%, or 98%) identical to SEQ ID NO: 87; d) an amino acid sequence having at least 70% (e.g., at least 80%, 90%, 95%, or 98%) identical to SEQ ID NO: 88; e) an amino acid sequence having at least 70% (e.g., at least 80%, 90%, 95%, or 98%) identical to SEQ ID NO: 89; f) an amino acid sequence having at least 70% (e.g., at least 80%, 90%, 95%, or 98%) identical to SEQ ID NO: 90; g) an amino acid sequence having at least 70% (e.g., at least 80%, 90%, 95%, or 98%) identical to SEQ ID NO: 91; h) an amino acid sequence having at least 70% (e.g., at least 80%, 90%, 95%, or 98%) identical to SEQ ID NO: 92; i) an amino acid sequence having at least 70% (e.g., at least 80%, 90%, 95%, or 98%) identical to SEQ ID NO: 93; j) an amino acid sequence having at least 70% (e.g., at least 80%, 90%, 95%, or 98%) identical to SEQ ID NO: 94; k) an amino acid sequence having at least 70% (e.g., at least 80%, 90%, 95%, or 98%) identical to SEQ ID NO: 95; l) an amino acid sequence having at least 70% (e.g., at least 80%, 90%, 95%, or 98%) identical to SEQ ID NO: 96; m) an amino acid sequence having at least 70% (e.g., at least 80%, 90%, 95%, or 98%) identical to SEQ ID NO: 97; n) an amino acid sequence having at least 70% (e.g., at least 80%, 90%, 95%, or 98%) identical to SEQ ID NO: 98; o) an amino acid sequence having at least 70% (e.g., at least 80%, 90%, 95%, or 98%) identical to SEQ ID NO: 99; p) an amino acid sequence having at least 70% (e.g., at least 80%, 90%, 95%, or 98%) identical to SEQ ID NO: 100; q) an amino acid sequence having at least 70% (e.g., at least 80%, 90%, 95%, or 98%) identical to SEQ ID NO: 101; r) an amino acid sequence having at least 70% (e.g., at least 80%, 90%, 95%, or 98%) identical to SEQ ID NO: 102; or s) an amino acid sequence having at least 70% (e.g., at least 80%, 90%, 95%, or 98%) identical to SEQ ID NO: 103.

The present disclosure provides a method of producing a doubled haploid inducer (DHI) line, the method comprising a) providing a transformed first haploid inducer line comprising a heterologous DNA construct comprising a genetic chromosome doubling agent; b) providing a second haploid inducer line containing a marker; c) crossing the first haploid inducer line with the second haploid inducer line to produce a combination of maternal and diploid embryos; d) selecting the maternal embryos based on the absence of the selectable marker from the second haploid inducer line; and e) obtaining a double haploid inducer (DHI) line comprising the introduced genetic chromosome doubling agent, thereby producing the DHI line. In an aspect, the first haploid inducer comprises a loss-of-function mutation in a patatin-like phospholipase A2α gene. In an aspect, the loss-of-function mutation in the patatin-like phospholipase A2α gene is the MATRILINEAL (MA TL) gene. In an aspect, the marker is selected from a selectable marker, a reporter gene, a visible endogenous morphological marker, and combinations thereof. In an aspect, the selectable marker is selected from the group consisting of GUS, PMI, PAT, and combinations thereof. In an aspect, the reporter gene is selected from the group consisting of GFP, RFP, CFP, and combinations thereof. In an aspect, the visible endogenous morphological marker is selected from the group consisting of B1, R-nj, R1-scm, anthocyanin pigments, and combinations thereof. In an aspect, the genetic chromosome doubling agent is a genetic chromosome doubling polypeptide. In an aspect, the genetic chromosome doubling polypeptide is selected from the group consisting of a) a polypeptide allowing replication of a genome without cell division; b) a polypeptide destabilizing tubulin polymerization; c) a polypeptide altering cell cycle regulation; and d) combinations of the foregoing. In an aspect, the genetic chromosome doubling polypeptide allowing replication of the genome without cell division is selected from the group consisting of a) an amino acid sequence having at least 70% (e.g., at least 80%, 90%, 95%, or 98%) identical to SEQ ID NO: 24; b) an amino acid sequence having at least 70% (e.g., at least 80%, 90%, 95%, or 98%) identical to SEQ ID NO: 25; c) an amino acid sequence having at least 70% (e.g., at least 80%, 90%, 95%, or 98%) identical to SEQ ID NO: 26; or d) an amino acid sequence having at least 70% (e.g., at least 80%, 90%, 95%, or 98%) identical to SEQ ID NO: 2. In an aspect, the genetic chromosome doubling polypeptide destabilizing tubulin polymerization is selected from the group consisting of a) an amino acid sequence having at least 70% (e.g., at least 80%, 90%, 95%, or 98%) identical to SEQ ID NO: 3; orb) an amino acid sequence having at least 70% (e.g., at least 80%, 90%, 95%, or 98%) identical to SEQ ID NO: 28. In an aspect, the genetic chromosome doubling polypeptide altering cell cycle regulation is selected from the group consisting of a) an amino acid sequence having at least 70% (e.g., at least 80%, 90%, 95%, or 98%) identical to SEQ ID NO: 23; b) an amino acid sequence having at least 70% (e.g., at least 80%, 90%, 95%, or 98%) identical to SEQ ID NO: 86; c) an amino acid sequence having at least 70% (e.g., at least 80%, 90%, 95%, or 98%) identical to SEQ ID NO: 87; d) an amino acid sequence having at least 70% (e.g., at least 80%, 90%, 95%, or 98%) identical to SEQ ID NO: 88; e) an amino acid sequence having at least 70% (e.g., at least 80%, 90%, 95%, or 98%) identical to SEQ ID NO: 89; f) an amino acid sequence having at least 70% (e.g., at least 80%, 90%, 95%, or 98%) identical to SEQ ID NO: 90; g) an amino acid sequence having at least 70% (e.g., at least 80%, 90%, 95%, or 98%) identical to SEQ ID NO: 91; h) an amino acid sequence having at least 70% (e.g., at least 80%, 90%, 95%, or 98%) identical to SEQ ID NO: 92; i) an amino acid sequence having at least 70% (e.g., at least 80%, 90%, 95%, or 98%) identical to SEQ ID NO: 93; j) an amino acid sequence having at least 70% (e.g., at least 80%, 90%, 95%, or 98%) identical to SEQ ID NO: 94; k) an amino acid sequence having at least 70% (e.g., at least 80%, 90%, 95%, or 98%) identical to SEQ ID NO: 95; l) an amino acid sequence having at least 70% (e.g., at least 80%, 90%, 95%, or 98%) identical to SEQ ID NO: 96; m) an amino acid sequence having at least 70% (e.g., at least 80%, 90%, 95%, or 98%) identical to SEQ ID NO: 97; n) an amino acid sequence having at least 70% (e.g., at least 80%, 90%, 95%, or 98%) identical to SEQ ID NO: 98; o) an amino acid sequence having at least 70% (e.g., at least 80%, 90%, 95%, or 98%) identical to SEQ ID NO: 99; p) an amino acid sequence having at least 70% (e.g., at least 80%, 90%, 95%, or 98%) identical to SEQ ID NO: 100; q) an amino acid sequence having at least 70% (e.g., at least 80%, 90%, 95%, or 98%) identical to SEQ ID NO: 101; r) an amino acid sequence having at least 70% (e.g., at least 80%, 90%, 95%, or 98%) identical to SEQ ID NO: 102; or s) an amino acid sequence having at least 70% (e.g., at least 80%, 90%, 95%, or 98%) identical to SEQ ID NO: 103.

The present disclosure provides a method of eliminating or reducing the step of exogenously inducing chromosome doubling after embryo formation in the production of an inbred population, the method comprising crossing a doubled haploid inducer (DHI) line comprising an introduced genetic chromosome doubling agent with a second line and obtaining a doubled haploid progeny of the second line without a separate step of chromosome doubling due to exogenous treatment of a chromosome doubling agent, wherein the double haploid progeny does not comprise the introduced genetic chromosome doubling agent. In an aspect, the doubled haploid inducer comprises a loss-of-function mutation in a patatin-like phospholipase A2α gene. In an aspect, the loss-of-function mutation in the patatin-like phospholipase A2α gene is the MATRILINEAL (MATL) gene. In an aspect, the doubled haploid inducer (DHI) line expresses a marker gene. In an aspect, wherein the marker gene is selected from a selectable marker, a reporter gene, a visible endogenous morphological marker, and combinations thereof. In an aspect, the selectable marker is selected from the group consisting of GUS, PMI, PAT, and combinations thereof. In an aspect, the reporter gene is selected from the group consisting of GFP, RFP, CFP, and combinations thereof. In an aspect, the visible endogenous morphological marker is selected from the group consisting of B1, R-nj, R1-scm, anthocyanin pigments, and combinations thereof. In an aspect, the introduced genetic chromosome doubling agent is a genetic chromosome doubling polypeptide. In an aspect, the genetic chromosome doubling polypeptide is selected from the group consisting of a) a polypeptide allowing replication of a genome without cell division; b) a polypeptide destabilizing tubulin polymerization; c) a polypeptide altering cell cycle regulation; and d) combinations of the foregoing. In an aspect, the genetic chromosome doubling polypeptide allowing replication of the genome without cell division is selected from the group consisting of a) an amino acid sequence having at least 70% (e.g., at least 80%, 90%, 95%, or 98%) identical to SEQ ID NO: 24; b) an amino acid sequence having at least 70% (e.g., at least 80%, 90%, 95%, or 98%) identical to SEQ ID NO: 25; c) an amino acid sequence having at least 70% (e.g., at least 80%, 90%, 95%, or 98%) identical to SEQ ID NO: 26; or d) an amino acid sequence having at least 70% (e.g., at least 80%, 90%, 95%, or 98%) identical to SEQ ID NO: 2. In an aspect, the genetic chromosome doubling polypeptide destabilizing tubulin polymerization is selected from the group consisting of a) an amino acid sequence having at least 70% (e.g., at least 80%, 90%, 95%, or 98%) identical to SEQ ID NO: 3; orb) an amino acid sequence having at least 70% (e.g., at least 80%, 90%, 95%, or 98%) identical to SEQ ID NO: 28. In an aspect, the genetic chromosome doubling polypeptide altering cell cycle regulation is selected from the group consisting of a) an amino acid sequence having at least 70% (e.g., at least 80%, 90%, 95%, or 98%) identical to SEQ ID NO: 23; b) an amino acid sequence having at least 70% (e.g., at least 80%, 90%, 95%, or 98%) identical to SEQ ID NO: 86; c) an amino acid sequence having at least 70% (e.g., at least 80%, 90%, 95%, or 98%) identical to SEQ ID NO: 87; d) an amino acid sequence having at least 70% (e.g., at least 80%, 90%, 95%, or 98%) identical to SEQ ID NO: 88; e) an amino acid sequence having at least 70% (e.g., at least 80%, 90%, 95%, or 98%) identical to SEQ ID NO: 89; f) an amino acid sequence having at least 70% (e.g., at least 80%, 90%, 95%, or 98%) identical to SEQ ID NO: 90; g) an amino acid sequence having at least 70% (e.g., at least 80%, 90%, 95%, or 98%) identical to SEQ ID NO: 91; h) an amino acid sequence having at least 70% (e.g., at least 80%, 90%, 95%, or 98%) identical to SEQ ID NO: 92; i) an amino acid sequence having at least 70% (e.g., at least 80%, 90%, 95%, or 98%) identical to SEQ ID NO: 93; j) an amino acid sequence having at least 70% (e.g., at least 80%, 90%, 95%, or 98%) identical to SEQ ID NO: 94; k) an amino acid sequence having at least 70% (e.g., at least 80%, 90%, 95%, or 98%) identical to SEQ ID NO: 95; l) an amino acid sequence having at least 70% (e.g., at least 80%, 90%, 95%, or 98%) identical to SEQ ID NO: 96; m) an amino acid sequence having at least 70% (e.g., at least 80%, 90%, 95%, or 98%) identical to SEQ ID NO: 97; n) an amino acid sequence having at least 70% (e.g., at least 80%, 90%, 95%, or 98%) identical to SEQ ID NO: 98; o) an amino acid sequence having at least 70% (e.g., at least 80%, 90%, 95%, or 98%) identical to SEQ ID NO: 99; p) an amino acid sequence having at least 70% (e.g., at least 80%, 90%, 95%, or 98%) identical to SEQ ID NO: 100; q) an amino acid sequence having at least 70% (e.g., at least 80%, 90%, 95%, or 98%) identical to SEQ ID NO: 101; r) an amino acid sequence having at least 70% (e.g., at least 80%, 90%, 95%, or 98%) identical to SEQ ID NO: 102; or s) an amino acid sequence having at least 70% (e.g., at least 80%, 90%, 95%, or 98%) identical to SEQ ID NO: 103.

The present disclosure provides a method of maintaining a doubled haploid inducer (DHI) line comprising a genetic chromosome doubling agent, the method comprising a) providing a maintainer line comprising a heterologous maintainer construct, the maintainer construct comprising (i) a repressor or an inhibitor component for the genetic chromosome doubling agent, (ii) a pollen transmission prevention component, and (iii) a selectable marker component to identify seeds that contain the maintainer construct; b) self-fertilizing the maintainer line having the maintainer construct and the genetic chromosome doubling agent; c) identifying seeds containing the maintainer construct from the self-fertilization, wherein the maintainer construct is present in about 50% of the F1 seeds; and d) growing the seeds containing the maintainer construct, thereby maintaining the DHI line in a diploid state for further seed increases or haploid induction crosses. In an aspect, the repressor or the inhibitor is a transcriptional repressor of the genetic chromosome doubling agent. In an aspect, the repressor or the inhibitor is an artificial micro RNA targeting the transcript of the genetic chromosome doubling agent. In an aspect, the selectable marker is a color-based marker suitable for seed sorting. In an aspect, the pollen transmission prevent component is a genetic element that reduces pollen viability thereby preventing the transmission of the maintainer construct through the pollen.

The present disclosure provides a doubled haploid inducer (DHI) maintainer line comprising a genetic chromosome doubling agent and a heterologous maintainer construct, the maintainer construct comprising (i) a repressor or an inhibitor component for the genetic chromosome doubling agent, (ii) a pollen transmission prevention component, and (iii) a selectable marker component to identify seeds that contain the maintainer construct. In an aspect, the repressor or the inhibitor is a transcriptional repressor of the genetic chromosome doubling agent. In an aspect, the repressor or the inhibitor is an artificial micro RNA targeting the transcript of the genetic chromosome doubling agent. In an aspect, the selectable marker is a color-based marker suitable for seed sorting. In an aspect, the pollen transmission prevent component is a genetic element that reduces pollen viability thereby preventing the transmission of the maintainer construct through the pollen.

### DESCRIPTION OF THE FIGURES

FIG. 1 shows construct designs useful in the methods of the present disclosure.

FIG. 2 shows a schematic representation of the methods of the present disclosure.

FIG. 3 shows a schematic using a doubled haploid inducer produced by the methods disclosed herein.

FIG. 4 shows a schematic representation of the methods of the present disclosure.

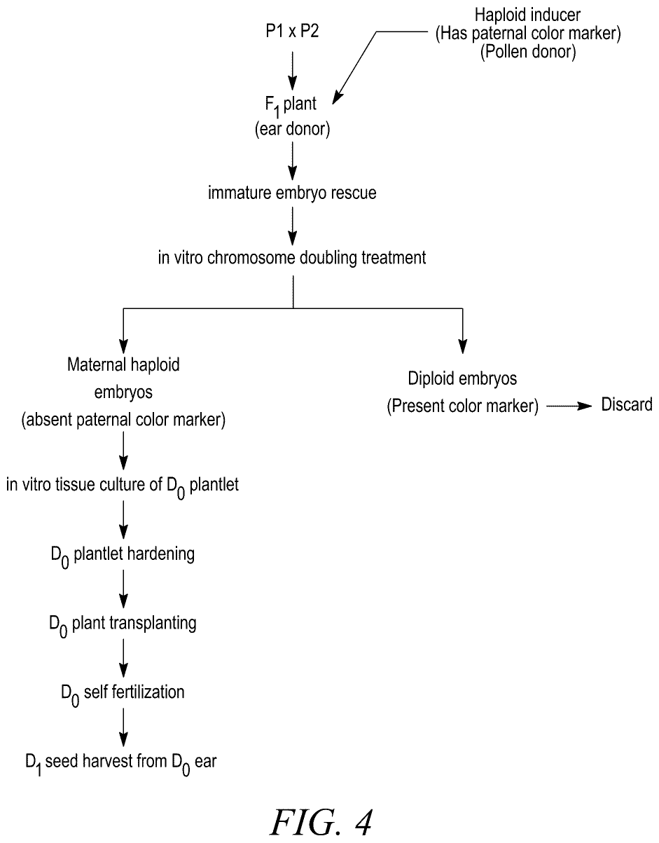

FIG. 5 shows a schematic of a maintainer construct (A), the genetic chromosome doubling agent (B), and seed identification (C) for maintaining the doubled haploid inducer line for seed increase and double haploid induction purposes.

### DETAILED DESCRIPTION

The disclosures herein will be described more fully hereinafter with reference to the accompanying figures, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods and compositions pertain having the benefit of the teachings presented in the following descriptions and the associated figures. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” includes the aspect of “consisting of.” Unless defined otherwise, all technical and scientific terms used herein have the same meaning as commonly understood by one of ordinary skill in the art to which the disclosed methods and compositions belong. In this specification and in the claims which follow, reference will be made to a number of terms which shall be defined herein.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

In plants, germ line cells (germline) provide the transgenerational inheritance of genetic information in each subsequent generation by producing spore mother cells during sporogenesis. For example, sporogenesis provides the megaspore mother cell that develops the female gametes, the egg cell and central cell that give rise to the embryo and endosperm, respectively; or the microspore mother cell that develops the male gamete, giving rise to four haploid microspores, wherein each microspore further develops into a mature pollen grain. A key aspect for the unique role of germline cells is providing the genetic information a future offspring receives, wherein half of the genetic contribution is from the female gamete and half of the genetic contribution is from the male gamete. Fertilization of the egg cell with one sperm cell forms a diploid zygote, while a second sperm cells fuses with the two polar nuclei of the central cell to form a triploid endosperm. The endosperm is a terminally nourishing tissue for the embryo yet does not contribute to the germline. After fertilization, the zygote gives rise to an embryo, a process referred to as zygotic embryogenesis that is characteristic of sexual reproduction. A newly formed embryo undergoing such an embryogenesis developmental program comprising an underlying regulatory program affected by genetic determinants and epigenetic reprogramming leading from an embryogenic cell state to the acquisition of a differentiated cell fate, or cell fates, ultimately giving rise to a plant with all differentiated tissues thereof.

Parthenogenesis is a natural form of asexual reproduction wherein growth and development of female gametes (embryos) occur without fertilization by sperm. The female gamete produced parthenogenetically may be either haploid or diploid.

The methods of the present disclosure alter such developmental programs of plant sexual and asexual reproduction described above. Such methods are valuable as plant reproduction methods for agricultural use. The present disclosure provides methods using molecular mechanisms impacting the cell cycle that are useful for agricultural use and crop improvement.

When an inducer line is used to pollinate a diploid plant, haploid embryos are derived. One sperm nucleus from the pollen fuses with the polar nuclei in the embryo sac to create a triploid (3N) endosperm. The triploid endosperm will contain 2 sets of chromosomes from the female and 1 set of chromosomes from the male, which in this case is the inducer line. The haploid embryo contains a single set of chromosomes, which are derived from the female plant and which is doubled by the methods and compositions described herein.

Wide hybridization crosses can also be used to produce haploids, where pollen from a distant but related genus/species is used as an inducer cross. This method of haploid production occurs due to the elimination of the chromosomes from the pollinating parent. Methods and compositions described herein with respect to providing a genetic chromosome doubling agent can also be provided through a wide hybridization-based induction cross.

Parthenogenesis induction refers to a method of providing a stimulus to a cell that improves levels of maternal haploid induction. Apetala2 (AP2) variant peptides are used as parthenogenesis factors, specifically comprising polypeptides or polynucleotides encoding gene products for generating doubled haploids or haploid plants from female gametes. Maize female gametophytes contacted with a parthenogenesis factor gene product results in improved levels of maternal haploid induction. Specifically, the gametes of a maize plant develop into a haploid plant when the plant is transformed with a genetic construct including regulatory elements and structural genes capable of altering the cellular fate of the plant cells. Further, the gametes of a maize plant develop into a diploid plant when the plant is transformed with a genetic construct including regulatory elements and structural genes capable of altering cellular fate and cell cycle regulation of plant cells. In the methods of the present disclosure, cell cycle regulating proteins expressed from a genetic construct are used for altering cell fate and ploidy levels in vivo.

As used herein, a “parthenogenesis factor” includes, but is not limited to, gene products that improve levels of maternal haploid induction and asexual reproduction wherein growth and development of female gametes (embryos) occur without fertilization by sperm when expressed in egg cells.

As used herein, a “parthenogenesis treatment” is any of the treatments disclosed herein that elicits an parthenogenic response in the contacted cell.

As used herein, “asexual reproduction” means reproduction without the fusion of gametes.

As used herein, “central cell” means the female gamete giving rise to the endosperm.

As used herein, “egg cell” means the female gamete giving rise to the embryo.

As used herein, “megaspore mother cell” means the cell that develops into the female gametophyte, also known as a megasporocyte, or functional megaspore (FMS).

As used herein, “microspore mother cell” means the cell that develops into the male gametophyte, also known as a microsporocyte.

As used herein, “gametogenesis” means the development of gametophytes from spores.

As used herein, “parthenogenesis” means the formation of an embryo from an unfertilized egg cell.

As used herein, “pseudogamy” means the fertilization-dependent formation of endosperm from a central cell.

As used herein, “sexual reproduction” means the mode of reproduction whereby female (egg) and male (sperm) gametes fuse to form a zygote.

As used herein, “somatic embryogenesis” means the formation of an embryo from a sporophytic cell without gamete and seed formation.

As used herein, “sporogenesis” means the formation of spores from spore mother cells.

As used herein, “spore mother cell” means the first cell of the reproductive lineage, formed from sporophytic cells in female and male reproductive tissues of the plant.

As used herein, “vegetative reproduction” means a form of reproduction in which a new plant is formed without the formation of an embryo.

As used herein, the term “embryo” means embryos and progeny of the same, immature and mature embryos, immature zygotic embryo, zygotic embryos, somatic embryos, embryogenic callus, and embryos derived from mature ear-derived seed. An embryo is a structure that is capable of germinating to form a plant.

As used herein, “haploid” means a plant or a plant cell having a single set (genome) of chromosomes and the reduced number of chromosomes (n) is equal to that in the gamete.

As used herein, the term “1n” or “1n cell” means a cell containing a single set of chromosomes, typically the product of meiosis. Examples of a 1n cell include gametes such as sperm cells, egg cells, or tissues derived from a gamete through mitotic divisions, such as a 1n embryo or a 1n plant. In maize where the plant is normally diploid, and the gametes are haploid, such gamete-derived embryos or plants are referred to as haploid embryos and haploid plants.

As used herein, “diploid” means a plant or a plant cell having two sets (genomes) of chromosomes and the chromosome number (2n) is equal to that in the zygote.

As used herein, the term “2n” or “2n cell” means a cell containing two sets of chromosomes. Examples of 2n cells include a zygote, an embryo resulting from mitotic divisions of a zygote, or a plant produced by germination of a 2n embryo.

As used herein, “haploid plant” means a plant having a single set (genome) of chromosomes and the reduced number of chromosomes (n) is equal to that in the gamete.

As used herein, the term “diploid plant” means a plant having two sets (genomes) of chromosomes and the chromosome number (2n) is equal to that in the zygote.

As used herein, a “doubled haploid” or a “doubled haploid plant or cell” is one that is developed by the doubling of a haploid set of chromosomes, male or female. A plant or seed that is obtained from a doubled haploid plant that is selfed any number of generations may still be identified as a doubled haploid plant. A doubled haploid plant is considered a homozygous plant. A plant is a doubled haploid if it is fertile, even if the entire vegetative part of the plant does not consist of the cells with the doubled set of chromosomes. For example, a plant will be considered a doubled haploid plant if it contains viable gametes, even if it is chimeric.

As used herein, a “doubled haploid embryo” is an embryo that has one or more cells containing 2 sets of homozygous chromosomes that are then be grown into a doubled haploid plant.

As used herein, the term “clonal” means multiple propagated plant cells or plants that are genetically, epigenetically and morphologically identical.

As used herein, the term “gamete” means a 1n reproductive cell such as a sperm cell, an egg cell or an ovule cell resulting from meiosis.

As used herein, the term “haploid embryo” means a gamete-derived somatic structure.

As used herein, the term “somatic structure” means a tissue, organ or organism.

As used herein, the term “somatic cell” is a cell that is not a gamete. Somatic cells, tissues or plants are haploid, diploid, triploid, tetraploid, hexaploid, etc. A complete set of chromosomes is referred to as being 1n (haploid), with the number of chromosomes found in a single set of chromosomes being referred to as the monoploid number (x). For example, in the diploid plant Zea mays, 2n=2x=20 total chromosomes, while in diploid rice Oryza sativa, 2n=2x=24 total chromosomes. In a triploid plant, such as banana, 2n=3x=33 total chromosomes. In hexaploid wheat Triticum aestivum, 2n=6x=42. Ploidy levels also vary between cultivars within the same species, such as in sugarcane, Saccharum officinarum, where 2n=10x=80 chromosomes, but commercial sugarcane varieties range from 100 to 130 chromosomes.

As used herein “karyogamy” is the final step in the process of fusing together two haploid eukaryotic cells and refers specifically to the fusion of the two nuclei. Before karyogamy, each haploid cell has one complete copy of the organism's genome.

As used herein, the term “medium” includes compounds in a liquid state, a gaseous state, or a solid state.

As used herein, the term “selectable marker” means a marker that when expressed in a modified, transformed/transfected cell confers a phenotypic or genotypic characteristic that is distinguishable from a control sample.

As used herein, the term “EAR” means an “Ethylene-responsive element binding factor-associated Amphiphilic Repression motif” with a general consensus sequence of LLxLxL, DNLxxP, LxLxPP, R/KLFGV, or TLLLFR that act as transcriptional repression signals within transcription factors. Addition of an EAR-type repressor element to a DNA-binding protein such as a transcription factor, dCAS9, or LEXA (as examples) confers transcriptional repression function to the fusion protein (Kagale, S., and Rozwadowski, K. 2010. Plant Signaling and Behavior 5:691-694).

As used herein, the term “transcription factor” means a protein that controls the rate of transcription of specific genes by binding to the DNA sequence of the promoter and either up-regulating or down-regulating expression. Examples of transcription factors, which are also morphogenic genes, include members of the AP2/EREBP family (including the Babyboom (BBM) and Ovule Development Protein 2 (ODP2) genes and variants, plethora and aintegumenta sub-families, CAAT-box binding proteins such as LEC1 and HAP3, and members of the MYB, bHLH, NAC, MADS, bZIP and WRKY families. In an aspect, ZM-ODP2 (SEQ ID NO: 61 encoding SEQ ID NO: 62), Os-ODP2 (OsANT (Oryza sativa ANT, Genbank Accession NO. AP003313) ((SEQ ID NO: 63 encoding SEQ ID NO: 64)), and Os-ODP2 (Oryza sativa BMN, Genbank Accession NO. AY062180) ((SEQ ID NO: 65 encoding SEQ ID NO: 66))) are useful as morphogenic genes in the methods of the present disclosure.

As used herein, the term “synthetic transcription factor” refers to a molecule comprising at least two domains, a recognition domain and a regulatory domain not naturally occurring in nature.

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence also refers to RNA molecules such as micro-RNAs, interfering RNA or RNA hairpins, that when expressed down-regulate expression of an endogenous gene or another transgene.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated region).

As used herein, the term “transfer cassette” means a T-DNA comprising an expression cassette or expression cassettes flanked by the right border and the left border.

As used herein, the term “T-DNA” means a portion of a Ti plasmid that is inserted into the genome of a host plant cell.

As used herein, the term “embryogenesis factor” means a gene that when expressed enhances improved formation of a somatically-derived structure. More precisely, ectopic expression of an embryogenesis factor stimulates de novo formation of an organogenic structure, for example a structure from embryogenic callus tissue, that improves the formation of an embryo. This stimulated de novo embryogenic formation occurs either in the cell in which the embryogenesis factor is expressed, or in a neighboring cell. An embryogenesis factor gene is a transcription factor that regulates expression of other genes or a gene that influences hormone levels in a plant cell which stimulates embryogenic changes.

An embryogenesis factor is involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof.

In an aspect, the present disclosure provides a method for producing plants using asexual reproduction. Apogamy, a type of reproduction of flowering plants, is characterized by a diploid cell in the embryo sac developing into an embryo without being fertilized. Parthenogenesis is one form of apogamy and in a broader sense includes de novo embryogenic formation from a haploid gametophytic cell, for example an egg cell resulting from megasporogenesis.

The present disclosure provides efficient and effective methods of producing populations of recombinant inbred lines including, but not limited to, methods of enabling generation of doubled haploid recombinant populations.

A parthenogenesis factor may be used in combination with a morphogenic developmental gene involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or combinations thereof to improve maternal haploid production. When parthenogenesis factors are co-expressed with a morphogenic developmental gene improved methods for obtaining a maternal haploid plant are provided.

As used herein, the term “morphogenic gene” or “morphogenic developmental gene” means a gene that when ectopically expressed stimulates formation of a somatically-derived structure that produces a plant. More precisely, ectopic expression of the morphogenic gene stimulates the de novo formation of a somatic embryo or an organogenic structure, such as a shoot meristem, that produces a plant. This stimulated de novo formation occurs either in the cell in which the morphogenic gene is expressed, or in a neighboring cell. A morphogenic gene is a transcription factor that regulates expression of other genes, or a gene that influences hormone levels in a plant tissue, both of which stimulate morphogenic changes. A morphogenic gene may be stably incorporated into the genome of a plant or it may be transiently expressed. As used herein, the term “morphogenic factor” means a morphogenic gene and/or the protein expressed by a morphogenic gene.

Morphogenic genes involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof, such as WUS/WOX genes (WUS1, WUS2, WUS3, WOX2A, WOX4, WOX5, or WOX9) see U.S. Pat. Nos. 7,348,468 and 7,256,322 and United States Patent Application publications 2017/0121722 and 2007/0271628; Laux et al. (1996) Development 122:87-96; and Mayer et al. (1998) Cell 95:805-815; van der Graaff et al., 2009, Genome Biology 10:248; Dolzblasz et al. 2016. Mol. Plant 19:1028-39 are useful in the methods of the present disclosure. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS induces stem cells in vegetative tissues, which differentiate into somatic embryos (Zuo, et al. (2002) Plant J 30:349-359). Also of interest in this regard, would be a MYB118 gene (see U.S. Pat. No. 7,148,402), a MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963). Morphogenic genes useful in the present disclosure include, but are not limited to, functional WUS/WOX genes.

Morphogenic polynucleotide sequences and amino acid sequences of WUS/WOX homeobox polypeptides are useful in the disclosed methods. As defined herein, a “functional WUS/WOX nucleotide” or a “functional WUS/WOX gene” is any polynucleotide encoding a protein that contains a homeobox DNA binding domain, a WUS box, and an EAR repressor domain (Ikeda et al., 2009 Plant Cell 21:3493-3505). As demonstrated by Rodriguez et al., 2016 PNAS www.pnas.org/cgi/doi/10.1073/pnas.1607673113 removal of the dimerization sequence which leaves behind the homeobox DNA binding domain, a WUS box, and an EAR repressor domain results in a functional WUS/WOX polypeptide. The WUSCHEL protein, designated hereafter as WUS, plays a key role in the initiation and maintenance of the apical meristem, which contains a pool of pluripotent stem cells (Endrizzi et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a homeodomain protein which presumably functions as a transcriptional regulator (Mayer, et al., (1998) Cell The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In an aspect, the functional WUS/WOX homeobox polypeptide useful in the methods of the present disclosure is a WUS1, WUS2, WUS3, WOX2A, WOX4, WOX5, WOX5A, or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX homeobox polypeptide useful in the methods of the present disclosure is obtained from or derived from any plant. Functional WUS/WOX nucleotides encoding proteins that contain a homeobox DNA binding domain, a WUS box, and an EAR repressor domain useful in the methods of the present disclosure are disclosed in US Patent Application Publication Number 2020/0270622 incorporated herein by reference in its entirety.

Other morphogenic genes useful in the present disclosure include, but are not limited to, LEC1 (U.S. Pat. No. 6,825,397 incorporated herein by reference in its entirety, Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabiopsis AGL15 gene (Harding et al., 2003, Plant Physiol. 133:653-663), the FUSCA gene (Castle and Meinke, Plant Cell 6:25-41), and the PICKLE gene (Ogas et al., 1999, PNAS 96:13839-13844).

Generally, a pollen lethality polynucleotide or a non-transmission pollen polynucleotide indicates any polynucleotide that prevents the pollen from achieving fertilization. This non-transmissibility can be achieved at the transcript level (e.g., transcriptional impact) or at a protein level, e.g., enzymatic action resulting in pollen lethality or non-transmissibility. In certain embodiments, the pollen non-transmission is most effective if it occurs at the post-meiotic or gametophytic stage. The non-transmission pollen polynucleotide can work through various mechanisms. It may prevent the pollen from being viable for example by the expression of a toxic compound.

The present disclosure also includes plants obtained by any of the disclosed methods or compositions herein. The present disclosure also includes seeds from a plant obtained by any of the methods or compositions disclosed herein. As used herein, the term “plant” refers to whole plants, plant organs (e.g., leaves, stems, roots, etc.), plant tissues, plant cells, plant parts, seeds, propagules, embryos and progeny of the same. As used herein, the term “plant” refers to whole plants, plant organs (e.g., leaves, stems, roots, etc.), plant tissues, plant cells, plant parts, seeds, propagules, embryos and progeny of the same. Plant cells are differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature inflorescences, tassel, immature ear, silks, cotyledons, immature cotyledons, meristematic regions, callus tissue, cells from leaves, cells from stems, cells from roots, cells from shoots, gametophytes, sporophytes, pollen, microspores, multicellular structures (MCS), and embryo-like structures (ELS). Plant parts include differentiated and undifferentiated tissues including, but not limited to, roots, stems, shoots, leaves, pollen, seeds, tumor tissue and various forms of cells in culture (e. g., single cells, protoplasts, embryos, and callus tissue). The plant tissue may be in a plant or in a plant organ, tissue, or cell culture. Grain is intended to mean the mature seed produced by commercial growers for purposes other than growing or reproducing the species. Progeny, variants and mutants of the regenerated plants are also included within the scope of the present disclosure, provided these progeny, variants and mutants are derived from regenerated plants made using the methods and compositions disclosed herein and/or comprise the introduced polynucleotides disclosed herein.

As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. As used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic. A transgenic plant is defined as a mature, fertile plant that contains a transgene.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The compositions and methods of the present disclosure are applicable to a broad range of plant species, including dicotyledonous plants and monocotyledonous plants. Representative examples of plants that are treated in accordance with the methods disclosed herein include, but are not limited to, wheat, cotton, sunflower, safflower, tobacco, Arabidopsis, barley, oats, rice, maize, triticale, sorghum, rye, millet, flax, sugarcane, banana, cassava, common bean, cowpea, tomato, potato, beet, grape, Eucalyptus, wheat grasses, turf grasses, alfalfa, clover, soybean, peanuts, citrus, papaya, Setaria sp, cacao, cucumber, apple, Capsicum, bamboo, melon, ornamentals including commercial garden and flower bulb species, fruit trees, vegetable species, Brassica species, as well as interspecies hybrids. In a preferred embodiment, the compositions and methods of the present disclosure are applied to maize plants.

The methods of the present disclosure involve introducing a polypeptide, polynucleotide (i.e., DNA or RNA), or nucleotide construct (i.e., DNA or RNA) into a plant. As used herein, “introducing” means presenting to the plant the polynucleotide, polypeptide, or nucleotide construct in such a manner that the polynucleotide, polypeptide, or nucleotide construct gains access to the interior of a cell of the plant. The methods of the present disclosure do not depend on a particular method for introducing the polynucleotide, polypeptide, or nucleotide construct into a plant, only that the polynucleotide, polypeptide, or nucleotide construct gains access to the interior of at least one cell of the plant. Methods for introducing polynucleotides, polypeptides, or nucleotide constructs into plants include, but are not limited to, stable transformation methods, transient transformation methods and virus-mediated methods.

As used herein, a “stable transformation” is a transformation in which the polynucleotide or nucleotide construct introduced into a plant integrates into the genome of the plant and is capable of being inherited by the progeny thereof. “Transient transformation” means that a polynucleotide or nucleotide construct is introduced into the plant and does not integrate into the genome of the plant or a polypeptide is introduced into a plant. In addition, “transient”, in certain embodiments may represent the presence of an embryogenesis inducing agent in a cell where such an agent has been exogenously applied or secreted from a neighboring cell or is being produced from an extrachromosomal location (e.g., plasmid or another independently replicating origin), or not produced by a stably integrated recombinant DNA construct within the same cell.

As used herein, “contacting”, “comes in contact with” or “in contact with” mean “direct contact” or “indirect contact”. For example, cells are placed in a condition where the cells come into contact with any of the parthenogenesis inducing genes disclosed herein including, but not limited to, an embryogenesis inducing embryogenesis factor, a morphogenic developmental gene, a small molecule or a doubling agent. Such substance is allowed to be present in an environment where the cells survive (for example, medium or expressed in the cell or expressed in an adjacent cell) and act on the cells. For example, the medium comprising a doubling agent may have direct contact with the haploid cell or the medium comprising the doubling agent may be separated from the haploid cell by filter paper, plant tissues, or other cells thus the doubling agent is transferred through the filter paper or cells to the haploid cell.

As used herein, the term “biparental cross” is the cross-fertilization of two genetically different plants to obtain the first filial generation of offspring and/or any successive filial generation thereafter. As used herein a biparental cross includes the offspring that are the progeny of any filial generation of offspring, including cross-fertilizing an offspring to one of its parental lines or an individual genetically like its parent to obtain progeny with a genetic identity closer to that of the parent referred to as a “backcross” and/or any successive backcross generation thereafter.

The methods provided herein rely upon the use of bacteria-mediated and/or biolistic-mediated gene transfer to produce regenerable plant cells. Bacterial strains useful in the methods of the present disclosure include, but are not limited to, a disarmed Agrobacteria, an Ochrobactrum bacteria or a Rhizobiaceae bacteria (U.S. Pat. No. 9,365,859 incorporated herein by reference in its entirety). Standard protocols for particle bombardment (Finer and McMullen, 1991, In Vitro Cell Dev. Biol.—Plant 27:175-182), Agrobacterium-mediated transformation (Jia et al., 2015, Int J. Mol. Sci. 16:18552-18543; US2017/0121722 incorporated herein by reference in its entirety), or Ochrobactrum-mediated transformation (US2018/0216123 incorporated herein by reference in its entirety is used with the methods and compositions of the present disclosure. Numerous methods for introducing heterologous genes into plants are used to insert a polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant and include chemical transfection methods such as calcium phosphate, microorganism-mediated gene transfer such as Agrobacterium (Horsch, et al., (1985) Science 227:1229-31), Ochrobactrum (US2018/0216123), electroporation, micro-injection and biolistic bombardment. Expression cassettes and vectors and in vitro culture methods for plant cell or tissue transformation and regeneration of transgenic plants are available. See, e.g., Gruber, et al., “Vectors for Plant Transformation,” in Methods in Plant Molecular Biology and Biotechnology, supra, pp. 89-119.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), Ochrobactrum-mediated transformation (US2018/0216123), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (U.S. Pat. No. 6,825,397). See also, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Ishida, et al., (1996) Nature Biotechnology 14:745-750 (maize via Agrobacterium tumefaciens), all of which are herein incorporated by reference in their entirety. Methods and compositions for rapid plant transformation are also found in U.S. 2017/0121722, herein incorporated in its entirety by reference. Vectors useful in plant transformation are found in U.S. patent application Ser. No. 15/765,521, herein incorporated by reference in its entirety.

Reporter genes or selectable marker genes may also be included in the expression cassettes of the present disclosure. Examples of suitable reporter genes are found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other genes may be used the expression cassettes of the present disclosure that also assist in the recovery of transgenic events and include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

The present disclosure also provides methods of contacting haploid cells with an amount of a chromosome doubling agent before, during, after, or overlapping with any portion of the isolation and embryogenesis induction process used for generating a paternal gamete (androgenic) or a maternal gamete (gynogenic) doubled haploid population.

As used herein “recombinant” means a cell or vector, that has been modified by the introduction of a heterologous nucleic acid or a cell derived from a cell so modified. Thus, for example, a recombinant cell is a cell expressing a gene that is not found in identical form or location within the native (non-recombinant) cell or a cell that expresses a native gene in an expression pattern that is different from that of the native (non-recombinant) cell for example, the native gene is abnormally expressed, under expressed, has reduced expression or is not expressed at all because of deliberate human intervention. The term “recombinant” as used herein does not encompass the alteration of a cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette is incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

In an aspect, a regulatory element expressed in the egg cell of the plant is useful for regulating ZM-ODP2 peptide activity to induce maternal haploid induction, resulting in a percentage of the progeny produced being haploid (having half the number of chromosomes compared to the parent). In addition, alternative regulatory elements are used to further optimize parthenogenic maternal haploid induction levels. For example, regulatory elements such as those disclosed in US2015/0152430 (promoters including, but not limited to the AT-DD5 promoter, the AT-DD31 promoter, the AT-DD65 promoter, and the ZM-DD45) and those disclosed in US2018/0094273 (Zea mays egg cell promoters) are used in the methods of the present disclosure (US2015/0152430 and US2018/0094273 incorporated herein by reference in their entireties).

Cis regulatory elements are regulatory elements that affect gene expression. Cis regulatory elements are regions of non-coding DNA that regulate the transcription of neighboring genes, often as DNA sequences in the vicinity of the genes that they regulate. Cis regulatory elements typically regulate gene transcription by encoding DNA sequences conferring transcription factor binding.

As used herein “promoter” is an exemplary regulatory element and generally refers to a nucleotide sequence capable of controlling the expression of a coding sequence or functional RNA. In general, a coding sequence is located 3′ to a promoter sequence. The promoter sequence comprises proximal and more distal upstream elements, the latter elements are often referred to as enhancers. Accordingly, an “enhancer” is a nucleotide sequence that stimulates promoter activity and may be an innate element of the promoter or a heterologous element inserted to enhance the level or tissue-specificity of a promoter. Promoters may be derived in their entirety from a native gene or may be composed of different elements derived from different promoters found in nature, or even comprise synthetic nucleotide segments. Different regulatory elements may direct the expression of a gene in different tissues or cell types, or at different stages of development, or in response to different environmental conditions.

A “plant promoter” is a promoter capable of initiating transcription in plant cells. Exemplary plant promoters include, but are not limited to, those that are obtained from plants, plant viruses, and bacteria such as Agrobacterium or Rhizobium, which comprise genes expressed in plant cells. Examples are promoters that preferentially initiate transcription in certain tissues, such as leaves, roots, seeds, fibers, xylem vessels, tracheids or sclerenchyma. Such promoters are referred to as “tissue preferred” promoters. A “cell type” specific promoter primarily drives expression in certain cell types in one or more organs, for example, vascular cells in roots or leaves. An “inducible” or “regulatable” promoter is a promoter which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters are members of the class of “non-constitutive” promoters. A “constitutive” promoter is a promoter that causes a nucleic acid fragment to be expressed in most cell types at most times under most environmental conditions and states of development or cell differentiation.

Examples of pollen specific promoters that can be used include but are not limited to PG47 (Rogers et al. (1991), Pollen specific cDNA clones from Zea mays, Biochem. Biophys. Acta 1089, 411-413; Allen, R. L., and Lonsdale, D. M. (1992) Sequence analysis of three members of the maize polygalacturonase gene family expressed during pollen development, Plant Mol. Biol. 20, 343-345, Allen, R. L., and Lonsdale, D. M. (1993), Molecular characterization of one of the maize polygalacturonase gene family members which are expressed during late pollen development. The Plant Journal 3, 261-271); maize pollen-specific gene Zm13 (Hamilton et al. (1992) Plant Mol. Biol. 18:211-218; Guerrero et al. (1993) Mol. Gen. Genet. 224:161-168); microspore-specific promoters such as the apg gene promoter (Twell et al., Sex. Plant Reprod. 6:217-224 (1993)); further include a sunflower pollen-expressed gene SF3 (Baltz et al. (1992) The Plant Journal 2:713-721), B. napus pollen specific genes (Arnoldo et al. (1992) J. Cell. Biochem, Abstract No. Y101204). Such promoters are known in the art or can be discovered by known techniques; see, e.g., Bhalla and Singh (1999) Molecular control of male fertility in Brassica Proc. 10th Annual Rapeseed Congress, Canberra, Australia; van Tunen et al. (1990) Pollen-specific chi promoters from petunia: tandem promoter regulation of the chiA gene, Plant Cell 2:393-40; Jeon et al. (1999); and Twell et al. (1993) Activation and developmental regulation of an Arabidopsis anther-specific promoter in microspores and pollen of Nicotiana tabacum, Sex. Plant Reprod. 6:217-224.

In an aspect, egg cell promoters and egg cell specific promoters are useful in the methods of the present disclosure. In addition to those egg cell promoters and/or egg cell specific promoters disclosed herein and those disclosed in US2015/0152430 and US2018/0094273, each of which is incorporated herein in its entirety, egg cell promoters and/or egg cell specific promoters useful in the present disclosure include, but are not limited to the egg cell-specific EC1.1 and EC1.2 promoters disclosed in Sprunck et al., (2012) Science, 338, 1093-1097 and Steffen et al., (2007) Plant J., 51:281-92.

A “translation leader sequence” refers to a nucleotide sequence located between the promoter sequence of a gene and the coding sequence. The translation leader sequence is present in the fully processed mRNA upstream of the translation start sequence. The translation leader sequence may affect numerous parameters including, processing of the primary transcript to mRNA, mRNA stability and/or translation efficiency. Examples of translation leader sequences have been described (Turner and Foster (1995) Mol. Biotechnol. 3:225-236).

As used herein, “heterologous” refers to a nucleic acid that originates from a foreign species, or, if from the same species, is substantially modified from its native form in composition and/or genomic locus by deliberate human intervention. For example, a promoter operably linked to a heterologous structural gene that is from a species different from that from which the structural gene was derived, or, if from the same species, one or both are substantially modified from their original form and/or genomic location.

The parthenogenesis factors and morphogenic developmental genes useful in the methods of the present disclosure are provided in expression cassettes for expression in the plant of interest. The cassette includes 5′ and 3′ regulatory sequences operably linked to a parthenogenesis factor and morphogenic developmental gene sequence disclosed herein. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions (fusion proteins), by operably linked it is intended that the coding regions are in the same reading frame. The cassette may additionally contain at least one additional gene to be co-transformed into the organism. Alternatively, the parthenogenesis factor and morphogenic developmental gene(s) are provided on multiple expression cassettes. Such an expression cassette is provided with a plurality of restriction sites for insertion of the parthenogenesis factor and morphogenic developmental gene sequence to be under the transcriptional regulation of the regulatory regions (promoter(s)). The expression cassette may additionally contain selectable marker genes.

Polynucleotides useful in the methods of the present disclosure include, but are not limited to, parthenogenesis factors, morphogenic developmental genes, and cell cycle genes including Cyclin A, Cyclin B, Cyclin C, Cyclin D, Cyclin E, Cyclin F, Cyclin G, and Cyclin H; Pin1; E2F; Cdc25; RepA genes and similar plant viral polynucleotides encoding replication-associated proteins. See U.S. Patent Publication No. 2002/0188965 incorporated herein by reference in its entirety.

As used herein, a “chimeric gene expression cassette” is an expression cassette comprising a coding sequence operably linked to a transcription initiation region that is heterologous to the coding sequence and includes in the 5′-3′ direction of transcription, a transcriptional initiation region (i.e., a promoter) and translational initiation region, a secretion signal peptide, an embryogenesis inducing morphogenic developmental gene sequence, a fluorescent protein sequence, and a transcriptional and translational termination region (i.e., termination region) functional in plants.

The parthenogenesis inducing methods of the present disclosure improve maternal haploid embryo regeneration productivity and enable gene editing to provide regenerated gene-edited, maize maternal haploids.

In an aspect, haploid cells are contacted with an amount of a chromosome doubling agent to promote chromosome doubling followed by regenerating homozygous diploid plants from the treated haploid cells. The haploid microspore cells are in contact with the doubling agent before, during, or after initiation of microspore embryogenesis or embryo maturation. After chromosome doubling, the doubled haploid embryo will contain 2 copies of paternally derived chromosomes. The efficiency of the process for obtaining doubled haploid plants from haploid embryos may be greater than 10%, 20%, 30%, 50%, 60%, 70%, 80%, or 90%. The duration of contact between the haploid cells and the chromosomal doubling agent may vary. Contact may be from less than 24 hours, for example 4-12 hours, to about a week. The duration of contact is generally from about 8 hours to 2 days.

Methods of chromosome doubling are disclosed in Antoine-Michard, S. et al., Plant cell, tissue organ cult., Cordrecht, the Netherlands, Kluwer Academic Publishers, 1997, 48(3):203-207; Kato, A., Maize Genetics Cooperation Newsletter 1997, 36-37; and Wan, Y. et al., TAG, 1989, 77: 889-892. Wan, Y. et al., TAG, 1991, 81: 205-211. The disclosures of which are incorporated herein by reference. Typical doubling methods involve contacting the cells with colchicine, anti-microtubule agents or anti-microtubule herbicides, pronamide, nitrous oxide, or any mitotic inhibitor to create homozygous doubled haploid cells. The amount of colchicine used in medium is generally 0.01%-0.2% or approximately 0.05% of amiprophos-methyl (APM) (5-225 μM) may be used. The amount of colchicine ranges from approximately 400-600 mg/L or approximately 500 mg/L. The amount of pronamide in medium is approximately 0.5-20 μM. Examples of mitotic inhibitors are included in Table 1. Other agents may be used with the mitotic inhibitors to improve doubling efficiency. Such agents include dimethyl sulfoxide (DMSO), adjuvants, surfactants, and the like.

TABLE 1
Chemical chromosome doubling agents

| | | |
|---|---|---|
| Common Name/ | | |
| Trade name | CAS | IUPAC |

Colchicine and Colchicine Derivatives

| | |
|---|---|
| colchicine/ | (S)-N-(5,6,7,9-tetrahydro-1,2,3,10- |
| acetyltrimethylcolchicinic | tetramethoxy-9-oxobenzo (a) |
| acid | heptalen-7-yl) acetamide |
| colchicine derivatives | |

Carbamates

| | | |
|---|---|---|
| Carbetamide | (R)-1-(ethylcarbamoyl)ethyl | (2R)-N-ethyl-2- |
| | carbanilate | [[(phenylamino)carbonyl]oxy] |
| | | propanamide |
| chloropropham | | |
| Propham | | |

Benzamids

| | | |
|---|---|---|
| Pronamide/ | 3,5-dichloro-N-(1,1- | 3,5-dichloro-N-(1,1-dimethyl-2- |
| propyzamide | dimethylpropynyl)benzamide | propynyl)benzamide |
| Tebutam | | |

Benzoic Acids

| | | |
|---|---|---|
| Chlorthal dimethyl | | |
| (DCPA), | | |
| Dicamba/dianat/ | 3,6-dichloro-o-anisic acid | 3,6-dichloro-2-methoxybenzoic acid |
| disugran (dicamba- | | |
| methyl) (BANVEL, | | |
| CLARITY) | | |

Dinitroaniline chromosome doubling agents

| | | |
|---|---|---|
| benfluralin/benefin/ | N-butyl-N-ethyl-α,α,α- | N-butyl-N-ethyl-2,6-dinitro-4- |
| (BALAN) | trifluoro-2,6-dinitro-p- | (trifluoromethyl)benzenamine |
| | toluidine | |
| Butralin | (RS)-N-sec-butyl-4-tert- | 4-(1,1-dimethylethyl)-N-(1- |
| | butyl-2,6-dinitroaniline | methylpropyl)-2,6- |
| | | dinitrobenzenamine |
| Chloralin | | |
| dinitramine | N1,N1-diethyl-2,6-dinitro-4- | N3,N3-diethyl-2,4-dinitro-6- |
| | trifluoromethyl-m- | (trifluoromethyl)-1,3- |
| | phenylenediamine | benzenediamine |
| ethalfluralin | N-ethyl-α,α,α-trifluoro-N-(2- | N-ethyl-N-(2-methyl-2-propenyl)- |
| (Sonalan) | methylallyl)-2,6-dinitro-p- | 2,6-dinitro-4- |
| | toluidine | (trifluoromethyl)benzenamine |
| fluchloralin | N-(2-chloroethyl)-2,6- | N-(2-chloroethyl)-2,6-dinitro-N- |
| | dinitro-N-propyl-4- | propyl-4- |
| | (trifluoromethyl)aniline | (trifluoromethyl)benzenamine |
| | or | |
| | N-(2-chloroethyl)-α,α,α- | |
| | trifluoro-2,6-dinitro-N- | |
| | propyl-p-toluidine | |
| isopropalin | 4-isopropyl-2,6-dinitro-N,N- | 4-(1-methylethyl)-2,6-dinitro-N,N- |
| | dipropylaniline | dipropylbenzenamine |
| methalpropalin | α,α,α-trifluoro-N-(2- | N-(2-methyl-2-propeny1)-2,6- |
| | methylallyl)-2,6-dinitro-N- | dinitro-N-propyl-4- |
| | propyl-p-toluidine | (trifluoromethyl)benzenamine |
| nitralin | 4-methylsulfonyl-2,6-dinitro- | 4-(methylsulfonyl)-2,6-dinitro-N,N- |
| | N,N-dipropylaniline | dipropylbenzenamine |
| oryzalin | 3,5-dinitro-N4,N4- | 4-(dipropylamino)-3,5- |
| (SURFLAN) | dipropylsulfanilamide | dinitrobenzenesulfonamide |
| pendimethalin | N-(1-ethylpropyl)-2,6- | N-(1-ethylpropyl)-3,4-dimethyl-2,6- |
| (PROWL) | dinitro-3,4-xylidine | dinitrobenzenamine |
| prodiamine | 5-dipropylamino-α,α,α- | 2,4-dinitro-N3,N3-dipropyl-6- |
| | trifluoro-4,6-dinitro-o- | (trifluoromethyl)-1,3- |
| | toluidine | benzenediamine |
| | or | |
| | 2,6-dinitro-N1,N1-dipropyl- | |
| | 4-trifluoromethyl-m- | |
| | phenylenediamine | |
| profluralin | N-cyclopropylmethyl-α,α,α- | N-(cyclopropylmethyl)-2,6-dinitro- |
| | trifluoro-2,6-dinitro-N- | N-propyl-4- |
| | propyl-p-toluidine | (trifluoromethyl)benzenamine |
| | or | |
| | N-cyclopropylmethyl-2,6- | |
| | dinitro-N-propyl-4- | |
| | trifluoromethylaniline | |
| trifluralin (TREFLAN, | α,α,α-trifluoro-2,6-dinitro- | 2,6-dinitro-N,N-dipropy1-4- |
| TRIFIC, TRILLIN) | N,N-dipropyl-p-toluidine | (trifluoromethyl)benzenamine |

Phosphoroamidates

| | | |
|---|---|---|
| APM (Amiprofos | | |
| methyl); amiprophos- | | |
| methyl | | |
| Butamifos | O-ethyl O-6-nitro-m-tolyl | O-ethyl O-(5-methyl-2-nitrophenyl) |
| | (RS)-sec- | (1- |
| | butylphosphoramidothioate | methylpropyl)phosphoramidothioate |

Pyridines
Dithiopyr

| | | |
|---|---|---|
| Thiazopyr | methyl 2-difluoromethyl-5- | methyl 2-(difluoromethyl)-5-(4,5- |
| | (4,5-dihydro-1,3-thiazol-2- | dihydro-2-thiazoly1)-4-(2- |
| | yl)-4-isobutyl-6- | methylpropyl)-6-(trifluoromethyl)-3- |
| | trifluoromethylnicotinate | pyridinecarboxylate |

As an alternative to using chemical chromosome doubling agents, modulating expression of genes that impact the plant cell cycle (genetic chromosome doubling protein), either through stimulation of the cell cycle (and cell division) or through stimulation of endoreduplication is used to double the chromosome complement in an embryo. Increasing ploidy level in plant cells is achieved by modulating expression of genes that stimulate key control points in the cell cycle cell. It has been demonstrated that expression of parthenogenic factors using an egg cell promoter enhanced formation of maternal haploid embryos, while simultaneous expression of parthenogenic factors and ZM-DZ470 (a maize cyclin-D family member) not only resulted in maternal haploid embryo formation but also stimulated doubling of the chromosome number. Thus, the addition of cyclin-D over-expression in the forming maternal haploid embryo appears to provide an appropriate level of cell cycle stimulation to result in doubling of the 1n haploid chromosome number to 2n (diploid). It is expected that other plant genes that simulate the cell cycle (or cell division) in plants are useful to produce a similar doubling of the chromosome number in the forming maternal haploid embryos. It is expected that these genes will be useful in the methods of the present disclosure in providing genetic chromosome doubling activity to the maternal haploid embryo from the male gamete of the double haploid inducer. Examples of plant genes whose over-expression stimulates the cell cycle include cyclin-A in tobacco (Yu et al., 2003), cyclin-D in tobacco (Cockcroft et al., 2000, Nature 405:575-79; Schnittger et al., 2002, PNAS 99:6410-6415; Dewitte et al., 2003, Plant Cell 15:79-92)., E2FA in Arabidopsis (De Veylder et al., 2002, EMBO J 21:1360-1368), E2FB in Arabidopsis (Magyar et al., 2005, Plant Cell 17:2527-2541). Similarly, over-expression of viral genes that modulate plant cell cycle machinery are used, such as when over-expression of the Wheat Dwarf Virus RepA gene stimulates cell cycle progression (G1/S transition) and cell division in maize (Gordon-Kamm et al., 2002, PNAS 99:11975-11980). Conversely, plant genes whose encoded products inhibit the cell cycle have been shown to result in increased cell division when the gene, such as Cyclin-Dependent Kinase Inhibitor (ICK1/KRP), is down-regulated in Arabidopsis (Cheng et al 2013, Plant J 75:642-655). Thus, down-regulation of the KRP gene using an egg cell specific promoter to drive expression would be expected to have a similar effect as over-expression of DZ470, resulting in chromosome doubling. Methods of down-regulation of a gene such as KRP include expression of an artificial micro-RNA targeted to the KRP mRNA, or expression of a dCas9-repressor fusion that is targeted to the KRP promoter by a gRNA to that sequence. Finally, there are plant genes that specifically impact the process of endoreduplication. When using such genes, such as for example the ccs52gene or the Dell gene, in the methods of the present disclosure, it is expected that over-expression of ccs52 would result in an increased ploidy level as observed in Medicago sativa (Cebolla et al., 1999, EMBO J 18:4476-4484), and that down-regulation of Dell would result in an increased ploidy level as observed in Arabidopsis (Vlieghe et al., 2005, Current Biol 15:59-63). It is expected that other genes that stimulate the cell cycle, the G1/S transition, or endoreduplication are useful in the methods disclosed herein to increase ploidy level.

Repressor motifs, for example see Kagale and Rozwadowski (Epigenetics. 2011. 6: 141-146), are useful in the methods of the present disclosure. Ethylene-responsive element binding factor-associated Amphiphilic Repression (EAR) motif-mediated transcriptional repression, including EAR motifs defined by the consensus sequence patterns of either LxLxL and DLNxxP (see Hiratsu et al., 2003. Plant J. 35:177-192) are useful in the methods of the present disclosure. Of interest to the present disclosure are peptides including the amphiphilic repression motif disclosed in U.S. Pat. No. 9,499,831, incorporated herein by reference in its entirety, and the Dr1/DRAP1 global repressor complex (see U.S. Pat. No. 7,288,695 B2 incorporated herein by reference in its entirety), including the Dr1 motif that is similar to the motif found in Arabidopsis thaliana MYBL2 (see Matsui K, Umemura Y, Ohme-Takagi M. 2008. Plant J. 55:954-967).

Methods for creating haploid inducer lines, for example by ectopically expressing AP2 domain containing transcription factors. For example, preferably the method of Gordon-Kamm et al. was used (see U.S. Pat. No. 7,579,529; incorporated herein by reference in its entirety) are useful in the methods of the present disclosure.

Of interest to the current method is expression of the full length ZM-ODP2 peptide as described previously (see U.S. Pat. No. 7,579,529; incorporated herein by reference in its entirety) which induces parthenogenesis and embryo formation without fertilization when used in the methods disclosed herein. Additionally, the Pennisetum squamulatum AP2 transcription factor, Apospory-Specific-Genomic-Region BabyBoomLike (herein referred to as PsASGR-BBML) transgene induces parthenogenesis and embryo formation without fertilization. In maize, individuals with a PsASGR-BBML transgene fertilized with pollen having the R1-navajo anthocyanin color markers exhibited haploid embryo production (Steffen J G, et al. 2007. Plant J 51:281-292, US2016/0304901 A1, herein incorporated by reference in their entirety). More recently, the method of Khanday and Sundaresan demonstrated similar findings, for example in rice (see WO2018/098420 A1; incorporated herein by reference in its entirety).

Methods for the targeted insertion of a polynucleotide at a specific location in the plant genome are useful in the methods of the present disclosure. The insertion of the polynucleotide at a desired genomic location is achieved using a site-specific recombination system. See, for example, U.S. Pat. Nos. 6,187,994, 6,331,661, 6,262,341, 6,300,545 and 6,528,700, all of which are herein incorporated by reference in their entireties. Briefly, a polynucleotide of interest, flanked by two non-identical recombination sites, are contained in a T-DNA transfer cassette. The T-DNA transfer cassette is introduced into a plant having stably incorporated into its genome a target site which is flanked by two non-identical recombination sites that correspond to the sites of the transfer cassette. An appropriate recombinase is provided, and the transfer cassette is integrated at the target site. The polynucleotide of interest is thereby integrated at a specific chromosomal position in the plant genome.

The disclosed methods are used to introduce into explants polynucleotides that are useful to target a specific site for modification in the genome of a plant derived from the explant. Site specific modifications that are introduced with the disclosed methods include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods are used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods are used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence of the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods employing a guide polynucleotide/Cas endonuclease system to provide an effective system for modifying target sites within the genome of a cell and for editing a nucleotide sequence in the genome of a cell. Once a genomic target site is identified, a variety of methods are employed to further modify the target sites such that they contain a variety of polynucleotides of interest. The disclosed methods are used to introduce a CRISPR-Cas system for editing a nucleotide sequence in the genome of a cell. The nucleotide sequence to be edited (the nucleotide sequence of interest) is located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas gene includes a gene that is generally coupled, associated or close to or in the vicinity of flanking CRISPR loci. The terms “Cas gene” and “CRISPR-associated (Cas) gene” are used interchangeably herein.

In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease in which the ability to create a double-strand break is retained. Fragments and variants are obtained via methods such as site-directed mutagenesis and synthetic construction.

In an aspect, the Cas endonuclease gene is a plant codon optimized Streptococcus pyogenes Cas9 gene that recognizes any genomic sequence of the form N(12-30)NGG which is in principle targeted.

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity is used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that are used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities are fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

A “Dead-CAS9” (dCAS9) as used herein, is used to supply a transcriptional repressor domain. The dCAS9 has been mutated so that it no longer cuts DNA. The dCAS0 still binds when guided to a sequence by the gRNA and are also be fused to repressor elements. The dCAS9 fused to the repressor element, as described herein, is abbreviated to dCAS9˜REP, where the repressor element (REP) is any of the repressor motifs that have been characterized in plants. An expressed guide RNA (gRNA) binds to the dCAS9˜REP protein and targets the binding of the dCAS9-REP fusion protein to a specific predetermined nucleotide sequence within a promoter (a promoter within the T-DNA). For example, if this is expressed beyond—the border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the beyond-the-border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain is used in place of the gRNA and dCAS9˜REP (Urritia et al., 2003, Genome Biol. 4:231) as described above.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that interacts with a Cas endonuclease.

As used herein, the term “guide polynucleotide” relates to a polynucleotide sequence that forms a complex with a Cas endonuclease and enables the Cas endonuclease to recognize and optionally cleave a DNA target site. The guide polynucleotide is a single molecule or a double molecule. The guide polynucleotide sequence is a RNA sequence, a DNA sequence, or a combination thereof (a RNA-DNA combination sequence). Optionally, the guide polynucleotide comprises at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

Nucleotide sequence modification of the guide polynucleotide, VT domain and/or CER domain is selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the present disclosure the variable target domain is 12, 13, 14, 15, 16, 17, 18, 19, 20, 21, 22, 23, 24, 25, 26, 27, 28, 29 or 30 nucleotides in length.

In an aspect of the present disclosure, the guide nucleotide comprises a cRNA (or cRNA fragment) and a tracrRNA (or tracrRNA fragment) of the type II CRISPR/Cas system that forms a complex with a type II Cas endonuclease, wherein the guide nucleotide Cas endonuclease complex directs the Cas endonuclease to a plant genomic target site, enabling the Cas endonuclease to introduce a double strand break into the genomic target site. The guide nucleotide is introduced into a plant or plant cell directly using any method including, but not limited to, particle bombardment or topical applications.

In an aspect, the guide nucleotide is introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site is an endogenous site in the plant genome, or alternatively, the target site is heterologous to the plant and thereby not be naturally occurring in the genome, or the target site is found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant.

An “artificial target site” or “artificial target sequence” are used interchangeably herein and refer to a target sequence that has been introduced into the genome of a plant. Such an artificial target sequence is identical in sequence to an endogenous or native target sequence in the genome of a plant but is located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods are used to introduce into plants polynucleotides useful for gene suppression of a target gene in a plant. Reduction of the activity of specific genes (also known as gene silencing, or gene suppression) is desirable for several aspects of genetic engineering in plants. Techniques for gene silencing include, but not limited to antisense technology.

In an aspect, the disclosed methods are used to introduce into plants polynucleotides useful for the targeted integration of nucleotide sequences into a plant. For example, the disclosed methods are used to introduce T-DNA expression cassettes comprising nucleotide sequences of interest flanked by non-identical recombination sites are used to transform a plant comprising a target site. In an aspect, the target site contains at least a set of non-identical recombination sites corresponding to those on the T-DNA expression cassette. The exchange of the nucleotide sequences flanked by the recombination sites is affected by a recombinase. Thus, the disclosed methods are used for the introduction of T-DNA expression cassettes for targeted integration of nucleotide sequences, wherein the T-DNA expression cassettes which are flanked by non-identical recombination sites recognized by a recombinase that recognizes and implements recombination at the nonidentical recombination sites. Accordingly, the disclosed methods and composition are used to improve efficiency and speed of development of plants containing non-identical recombination sites.

Thus, the disclosed methods further comprise methods for the directional, targeted integration of exogenous nucleotides into a transformed plant. In an aspect, the disclosed methods use recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the T-DNA expression cassette.

It is recognized that the transformed plant prepared in this manner may comprise multiple target sites; i. e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system inserted into and functions in plant cells. The yeast FLP\FRT site specific recombination system has been shown to function in plants. To date, the system has been utilized for excision of unwanted DNA. See, Lyznik et at. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase is provided by any means including, but not limited to, providing to the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the present disclosure prevent or greatly suppress recombination between the two flanking recombination sites and excision of the nucleotide sequence contained therein. Accordingly, it is recognized that any suitable non-identical recombination sites may be utilized in the present disclosure, including FRT and mutant FRT sites, FRT and lox sites, lox and mutant lox sites.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about to about 50%, preferably less than about 10 to about 30%, more preferably less than about to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A. 80: 4223-4227. The FLP recombinase for use in the present disclosure may be that derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702. All of which are herein incorporated by reference. Such Cre sequence may also be synthesized using plant preferred codons.

Where appropriate, the nucleotide sequences to be inserted in the plant genome may be optimized for increased expression in the transformed plant. Where mammalian, yeast, or bacterial genes are used in the present disclosure, they are synthesized using plant preferred codons for improved expression. It is recognized that for expression in monocots, dicot genes are also synthesized using monocot preferred codons. See, for example, U.S. Pat. Nos. 5,380,831, and 5,436,391, and Murray et al. (1989) Nucleic Acids Res. 17: 477-498, herein incorporated by reference for methods of synthesizing plant preferred genes. The plant preferred codons may be determined from the codons utilized more frequently in the proteins expressed in the plant of interest. It is recognized that monocot or dicot preferred sequences may be constructed as well as plant preferred sequences for particular plant species. See, for example, EP 0359472; EP 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications that enhance gene expression in a cellular host are used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses FLP recombination target sites (FRT). The FRT has been identified as a minimal sequence comprising two 13 base pair repeats, separated by an eight (8) base spacer. The nucleotides in the spacer region are replaced with a combination of nucleotides, so long as the two 13-base repeats are separated by eight nucleotides. It appears that the actual nucleotide sequence of the spacer is not critical; however, for the practice of the present disclosure, some substitutions of nucleotides in the space region may work better than others. The eight-base pair spacer is involved in DNA-DNA pairing during strand exchange. The asymmetry of the region determines the direction of site alignment in the recombination event, which will subsequently lead to either inversion or excision. As indicated above, most of the spacer is mutated without a loss of function. See, for example, Schlake and Bode (1994) Biochemistry 33: 12746-12751, herein incorporated by reference.

FRT mutant sites are useful in the practice of the disclosed methods (see SEQ ID NO: 2, 3, 4 and 5 of U.S. Pat. No. 7,820,880, incorporated herein by reference in its entirety). Other mutant sites may be constructed by PCR-based mutagenesis for use in the methods of the present disclosure. The present disclosure is not restricted to the use of a particular FRT or recombination site, but rather that non-identical recombination sites or FRT sites are utilized for targeted insertion and expression of nucleotide sequences in a plant genome. Thus, other mutant FRT sites are constructed and utilized based upon the present disclosure.

As discussed above, bringing genomic DNA containing a target site with non-identical recombination sites together with a vector containing a T-DNA expression cassette with corresponding non-identical recombination sites, in the presence of the recombinase, results in recombination. The nucleotide sequence of the T-DNA expression cassette located between the flanking recombination sites is exchanged with the nucleotide sequence of the target site located between the flanking recombination sites. In this manner, nucleotide sequences of interest may be precisely incorporated into the genome of the host. Target sites are constructed having multiple non-identical recombination sites. Thus, multiple genes or nucleotide sequences are stacked or ordered at precise locations in the plant genome. Likewise, once a target site has been established within the genome, additional recombination sites may be introduced by incorporating such sites within the nucleotide sequence of the T-DNA expression cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a T-DNA expression cassette comprising a coding region, expression of the coding region will occur upon integration of the T-DNA expression cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing T-DNA expression cassettes as discussed above and selecting organisms with simple integration patterns. In the same manner, preferred sites within the genome are identified by comparing several transformation events. A preferred site within the genome includes one that does not disrupt expression of essential sequences and provides for adequate expression of the transgene sequence.

The following examples are offered by way of illustration and not by way of limitation.

### EXAMPLES

The aspects of the present disclosure are further defined in the following Examples, in which parts and percentages are by weight and degrees are Celsius, unless otherwise stated. These Examples, while indicating aspects of the present disclosure, are given by way of illustration only. From the above discussion and these Examples, one skilled in the art can ascertain the essential characteristics of the aspects of the present disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Plasmids

See Table 2 for a description of plasmids useful in the present disclosure.

TABLE 2

| SEQ | | |
|---|---|---|
| ID | | |
| NO: | Plasmid | Plasmid Elements |
| 29 | RV044054 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-BBM2 PRO:: ZM- |
| | | BBM2 5UTR::ZM-CYCD2::PINII TERM + UBI1ZM |
| | | PRO::UBI1ZM 5UTR(PHI)::UBI1ZM INTRON1(PHI)::Ds- |
| | | RED2::PINII TERM + LB |
| 30 | RX000002 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-BBM2 PRO:: ZM- |
| | | BBM2 5UTR::ZM-FIZZY-RELATED 2-like::PINII TERM + |
| | | UBI1ZM PRO::UBI1ZM 5UTR(PHI)::UBI1ZM |
| | | INTRON1(PHI)::Ds-RED2::PINII TERM + LB |
| 31 | RX000003 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-BBM2 PRO:: ZM- |
| | | BBM2 5UTR::ZM-FIZZY-RELATED 2-like B::PINII TERM + |
| | | UBI1ZM PRO::UBI1ZM 5UTR(PHI)::UBI1ZM |
| | | INTRON1(PHI)::Ds-RED2::PINII TERM + LB |
| 32 | RX000004 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-BBM2 PRO:: ZM- |
| | | BBM2 5UTR::ZM-FIZZY-RELATED 2-like C::PINII TERM + |
| | | UBI1ZM PRO::UBI1ZM 5UTR(PHI)::UBI1ZM |
| | | INTRON1(PHI)::Ds-RED2::PINII TERM + LB |
| 33 | RX000005 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-BBM2 PRO::ZM- |
| | | BBM2 5UTR::ZM-FIZZY-RELATED 2-like D::PINII TERM + |
| | | UBI1ZM PRO::UBI1ZM 5UTR(PHI)::UBI1ZM |
| | | INTRON1(PHI)::Ds-RED2::PINII TERM + LB |
| 34 | RX000006 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-BBM2 PRO::ZM- |
| | | BBM2 5UTR::tubulin-PS00227minus::PINII TERM + UBI1ZM |
| | | PRO::UBI1ZM 5UTR(PHI)::UBI1ZM INTRON1(PHI)::Ds- |
| | | RED2::PINII TERM + LB |
| 35 | RV042024 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PRF2 PRO:: ZM-PRF2 |
| | | 5UTR:: ZM-CYCD2::PINII TERM + UBI1ZM PRO::UBI1ZM |
| | | 5UTR(PHI)::UBI1ZM INTRON1(PHI)::Ds-RED2::PINII TERM + LB |
| 36 | RX000007 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PRF2 PRO:: ZM-PRF2 |
| | | 5UTR:: ZM-FIZZY-RELATED 2-like::PINII TERM + UBI1ZM |
| | | PRO::UBI1ZM 5UTR(PHI)::UBI1ZM INTRON1(PHI)::Ds- |
| | | RED2::PINII TERM + LB |
| 37 | RX000008 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PRF2 PRO:: ZM-PRF2 |
| | | 5UTR:: ZM-FIZZY-RELATED 2-like B::PINII TERM + UBI1ZM |
| | | PRO::UBI1ZM 5UTR(PHI)::UBI1ZM INTRON1(PHI)::Ds- |
| | | RED2::PINII TERM + LB |
| 38 | RX000009 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PRF2 PRO:: ZM-PRF2 |
| | | 5UTR:: ZM-FIZZY-RELATED 2-like C::PINII TERM + UBI1ZM |
| | | PRO::UBI1ZM 5UTR(PHI)::UBI1ZM INTRON1(PHI)::Ds- |
| | | RED2::PINII TERM + LB |
| 39 | RX000010 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB 1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PRF2 PRO::ZM-PRF2 |
| | | 5UTR:: ZM-FIZZY-RELATED 2-like D::PINII TERM + UBI1ZM |
| | | PRO::UBI1ZM 5UTR(PHI)::UBI1ZM INTRON1(PHI)::Ds- |
| | | RED2::PINII TERM + LB |
| 40 | RX000011 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PRF2 PRO:: ZM-PRF2 |
| | | 5UTR::tubulin-PS00227minus::PINII TERM + UBI1ZM |
| | | PRO::UBI1ZM 5UTR(PHI)::UBI1ZM INTRON1 (PHI)::Ds- |
| | | RED2::PINII TERM + LB |
| 41 | RV042025 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-SF3 PRO::ZM-SF3 |
| | | 5UTR:: ZM-CYCD2::PINII TERM + UBI1ZM PRO::UBI1ZM |
| | | 5UTR(PHI)::UBI1ZM INTRON1(PHI)::Ds-RED2::PINII TERM + LB |
| 42 | RX000012 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-SF3 PRO:: ZM-SF3 |
| | | 5UTR:: ZM-FIZZY-RELATED 2-like::PINII TERM + UBI1ZM |
| | | PRO::UBI1ZM 5UTR(PHI)::UBI1ZM INTRON1(PHI)::Ds- |
| | | RED2::PINII TERM + LB |
| 43 | RX000013 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-SF3 PRO:: ZM-SF3 |
| | | 5UTR:: ZM-FIZZY-RELATED 2-like B::PINII TERM + UBI1ZM |
| | | PRO::UBI1ZM 5UTR(PHI)::UBI1ZM INTRON1(PHI)::Ds- |
| | | RED2::PINII TERM + LB |
| 44 | RX000014 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-SF3 PRO::ZM-SF3 |
| | | 5UTR:: ZM-FIZZY-RELATED 2-like C::PINII TERM + UBI1ZM |
| | | PRO::UBI1ZM 5UTR(PHI)::UBI1ZM INTRON1(PHI)::Ds- |
| | | RED2::PINII TERM + LB |
| 45 | RX000015 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-SF3 PRO:: ZM-SF3 |
| | | 5UTR:: ZM-FIZZY-RELATED 2-like D::PINII TERM + UBI1ZM |
| | | PRO::UBI1ZM 5UTR(PHI)::UBI1ZM INTRON1(PHI)::Ds- |
| | | RED2::PINII TERM + LB |
| 46 | RX000016 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-SF3 PRO:: ZM-SF3 |
| | | 5UTR:: tubulin-PS00227minus::PINII TERM + UBI1ZM |
| | | PRO::UBI1ZM 5UTR(PHI)::UBI1ZM INTRON1 (PHI)::Ds- |
| | | RED2::PINII TERM + LB |
| 47 | RV042026 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PLAA PRO (TR1):: |
| | | ZM-PLAA 5UTR::ZM-CYCD2::PINII TERM + UBI1ZM |
| | | PRO::UBI1ZM 5UTR(PHI)::UBI1ZM INTRON1(PHI)::Ds- |
| | | RED2::PINII TERM + LB |
| 48 | RX000017 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PLAA PRO (TR1):: |
| | | ZM-PLAA 5UTR:: ZM-FIZZY-RELATED 2-like::PINII TERM + |
| | | UBI1ZM PRO::UBI1ZM 5UTR(PHI)::UBI1ZM |
| | | INTRON1(PHI)::Ds-RED2::PINII TERM + LB |
| 49 | RX000018 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PLAA PRO (TR1):: |
| | | ZM-PLAA 5UTR:: ZM-FIZZY-RELATED 2-like B::PINII TERM + |
| | | UBI1ZM PRO::UBI1ZM 5UTR(PHI)::UBI1ZM |
| | | INTRON1(PHI)::Ds-RED2::PINII TERM + LB |
| 50 | RX000019 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PLAA PRO (TR1):: |
| | | ZM-PLAA 5UTR:: ZM-FIZZY-RELATED 2-like C::PINII TERM + |
| | | UBI1ZM PRO::UBI1ZM 5UTR(PHI)::UBI1ZM |
| | | INTRON1(PHI)::Ds-RED2::PINII TERM + LB |
| 51 | RX000020 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PLAA PRO (TR1):: |
| | | ZM-PLAA 5UTR:: ZM-FIZZY-RELATED 2-like D::PINII TERM + |
| | | UBI1ZM PRO::UBI1ZM 5UTR(PHI)::UBI1ZM |
| | | INTRON1(PHI)::Ds-RED2::PINII TERM + LB |
| 52 | RX000021 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + ZM-PLAA PRO (TR1):: |
| | | ZM-PLAA 5UTR:: tubulin-PS00227minus::PINII TERM + UBI1ZM |
| | | PRO::UBI1ZM 5UTR(PHI)::UBI1ZM INTRON1 (PHI)::Ds- |
| | | RED2::PINII TERM + LB |
| 67 | RV044484 | RB + SB-ALS PRO::ZM-ALS (HRA) EXON1::ST-LS1 INTRON2 |
| | | FRAG1 + LOXP + ZM-AXIG1 1XOP-B PRO-V1::ZM-WUS2 |
| | | (CDNA)::IN2-1 TERM + AT-5-IV-2 INS + ATTB1 + ZM-PLTP |
| | | PRO::ZM-PLTP 5 UTR::ZM-ODP2 (ALT1)::OS-T28 TERM::PINII |
| | | TERM::CZ19B1 TERM + ALL STOPS2 + ZM-GLB1 PRO::MO- |
| | | CRE EXON1::ST-LS1 INTRON2-V2::MO-CRE EXON2::PINII |
| | | TERM-V3 + SB-UBI PRO::SB-UBI INTRON1::ZS-GREEN1::OS- |
| | | UBI TERM + MINI-ALLSTOPS + LOXP + ZM-ALS (HRA) |
| | | EXON2::SB-PEPC1 TERM (MOD1) + OS-BBM1 PRO(3KB)::ZM- |
| | | CYCD2::PINII TERM + UBI1ZM PRO::UBI1ZM |
| | | 5UTR(PHI)::UBI1ZM INTRON1(PHI)::Ds-RED2::PINII TERM + LB |

### Example 2: Culture Media

See Table 3 and Table 4 for a description of media formations for transformation, selection and regeneration useful in the methods of the present disclosure.

TABLE 3

| | Units | | | | |
|---|---|---|---|---|---|
| | per | | | | |
| Medium components | liter | 12R | 810K | 700A | 710I |
| MS BASAL SALT | g | | | 4.3 | 4.3 |
| MIXTURE | | | | | |
| N6 BASAL SALTS | g | | | | |
| N6 MACRONUTRIENTS | ml | | | | |
| 10X | | | | | |
| POTASSIUM NITRATE | g | | | | |
| B5H MINOR SALTS 1000X | ml | | | | |
| NaFe EDTA FOR B5H | ml | | | | |
| 100X | | | | | |
| ERIKSSON'S VITAMINS | ml | | | | |
| 1000X | | | | | |
| S&H VITAMIN STOCK | ml | | | | |
| 100X | | | | | |
| THIAMINE•HCL | mg | | | 10.0 | 10.0 |
| L-PROLINE | g | | | | 0.7 |
| CASEIN HYDROLYSATE | g | | | | |
| (ACID) | | | | | |
| SUCROSE | g | | | 68.5 | 20.0 |
| GLUCOSE | g | 5.0 | | 36.0 | 10.0 |
| 2,4-D | mg | | | 1.5 | 2.0 |
| AGAR | g | 15.0 | | | 8.0 |
| BACTO-AGAR | g | | 15.0 | | |
| DICAMBA | g | | | | |
| SILVER NITRATE | mg | | | | |
| AGRIBIO Carbenicillin | mg | | | | |
| Timentin | mg | | | | |
| Cefotaxime | mg | | | | |
| MYO-INOSITOL | g | | | 0.1 | 0.1 |
| NICOTINIC ACID | mg | | | 0.5 | 0.5 |
| PYRIDOXINE•HCL | mg | | | 0.5 | 0.5 |
| VITAMIN ASSAY | g | | | 1.0 | |
| CASAMINO ACIDS | | | | | |
| MES BUFFER | g | | | | 0.5 |
| ACETOSYRINGONE | uM | | | | 100.0 |
| ASCORBIC ACID | mg | | | | 10.0 |
| 10 MG/ML (7S) | | | | | |
| MS VITAMIN STOCK | ml | | | | |
| SOL. | | | | | |
| ZEATIN | mg | | | | |
| CUPRIC SULFATE | mg | | | | |
| IAA 0.5 MG/ML (28A) | ml | | | | |
| ABA 0.1 mm | ml | | | | |
| THIDIAZURON | mg | | | | |
| AGRIBIO Carbenicillin | mg | | | | |
| BAP | mg | | | | |
| YEAST EXTRACT (BD | g | | 5.0 | | |
| Difco) | | | | | |
| PEPTONE | g | | 10.0 | | |
| SODIUM CHLORIDE | g | | 5.0 | | |
| SPECTINOMYCIN | mg | 50.0 | 50.0 | | |
| FERROUS SULFATE•7H20 | ml | 2.0 | | | |
| AB BUFFER 20X (12D) | ml | 50.0 | | | |
| AB SALTS 20X (12E) | ml | 50.0 | | | |
| THYMIDINE | mg | 50.0 | 50.0 | 50.0 | |
| GENTAMYCIN | mg | 50.0 | 50.0 | | |
| MEROPENEM | mg | | | | |
| pH | | | 6.8 | 5.2 | 5.8 |

TABLE 4

| | Units | | | | |
|---|---|---|---|---|---|
| | per | | | | |
| Medium components | liter | 605J | 605B | 562V | 289Q |
| MS BASAL SALT | g | 4.3 | 4.3 | | 4.3 |
| MIXTURE | | | | | |
| N6 BASAL SALTS | g | | | 4.0 | |
| N6 MACRONUTRIENTS | ml | 60.0 | 60.0 | | |
| 10X | | | | | |
| POTASSIUM NITRATE | g | 1.7 | 1.7 | | |
| B5H MINOR SALTS 1000X | ml | 0.6 | 0.6 | | |
| NaFe EDTA FOR B5H | ml | 6.0 | 6.0 | | |
| 100X | | | | | |
| ERIKSSON'S VITAMINS | ml | 0.4 | 0.4 | 1.0 | |
| 1000X | | | | | |
| S&H VITAMIN STOCK | ml | 6.0 | 6.0 | | |
| 100X | | | | | |
| THIAMINE•HCL | mg | 0.5 | 0.5 | 0.5 | |
| L-PROLINE | g | 2.0 | 2.0 | 0.69 | 0.7 |
| CASEIN HYDROLYSATE | g | 0.3 | 0.3 | | |
| (ACID) | | | | | |
| SUCROSE | g | 20.0 | 20.0 | 30.0 | 60.0 |
| GLUCOSE | g | 0.6 | 0.6 | | |
| 2,4-D | mg | 0.8 | 0.8 | 2.0 | |
| AGAR | g | 6.0 | 6.0 | 8.0 | 8.0 |
| BACTO-AGAR | g | | | | |
| DICAMBA | g | 1.2 | 1.2 | | |
| SILVER NITRATE | mg | 3.4 | 3.4 | 0.85 | |
| AGRIBIO Carbenicillin | mg | 100.0 | | | |
| Timentin | mg | | | | 150.0 |
| Cefotaxime | mg | | | | 100.0 |
| MYO-INOSITOL | g | | | | 0.1 |
| NICOTINIC ACID | mg | | | | |
| PYRIDOXINE•HCL | mg | | | | |
| VITAMIN ASSAY | g | | | | |
| CASAMINO ACIDS | | | | | |
| MES BUFFER | g | | | | |
| ACETOSYRINGONE | uM | | | 100.0 | |
| ASCORBIC ACID | mg | | | | |
| 10 MG/ML (7S) | | | | | |
| MS VITAMIN STOCK | ml | | | | 5.0 |
| SOL. | | | | | |
| ZEATIN | mg | | | | 0.5 |
| CUPRIC SULFATE | mg | | | | 1.3 |
| IAA 0.5 MG/ML (28A) | ml | | | | 2.0 |
| ABA 0.1 mm | ml | | | | 1.0 |
| THIDIAZURON | mg | | | | 0.1 |
| AGRIBIO Carbenicillin | mg | | | | 100.0 |
| BAP | mg | | | | 1.0 |
| YEAST EXTRACT (BD | g | | | | |
| Difco) | | | | | |
| PEPTONE | g | | | | |
| SODIUM CHLORIDE | g | | | | |
| SPECTINOMYCIN | mg | | | | |
| FERROUS SULFATE•7H20 | ml | | | | |
| AB BUFFER 20X (12D) | ml | | | | |
| AB SALTS 20X (12E) | ml | | | | |
| THYMIDINE | mg | | | 50.0 | |
| GENTAMYCIN | mg | | | | |
| MEROPENEM | mg | | 10.0 | | |
| pH | | 5.8 | 5.8 | 5.8 | 5.6 |

### Example 3: Agrobacterium-Mediated Transformation of Corn

### A. Preparation of Agrobacterium Master Plate

Agrobacterium tumefaciens harboring a binary donor vector was streaked out from a −80° C. frozen aliquot onto solid 12R medium and cultured at 28° C. in the dark for 2-3 days to make a master plate.

### B. Growing Agrobacterium on Solid Medium

A single colony or multiple colonies of Agrobacterium were picked from the master plate and streaked onto a second plate containing 810K medium and incubated at 28° C. in the dark overnight. Agrobacterium infection medium (700A; 5 ml) and 100 mM 3′-5′-Dimethoxy-4′-hydroxyacetophenone (acetosyringone; 5 μL) were added to a 14-mL conical tube in a hood. About 3 full loops of Agrobacterium from the second plate were suspended in the tube and the tube was then vortexed to make an even suspension. The suspension (1 ml) was transferred to a spectrophotometer tube and the optical density (550 nm) of the suspension was adjusted to a reading of about 0.35-1.0. The Agrobacterium concentration was approximately 0.5 to 2.0×10⁹ cfu/mL. The final Agrobacterium suspension was aliquoted into 2 mL microcentrifuge tubes, each containing about 1 mL of the suspension. The suspensions were then used as soon as possible.

### C. Growing Agrobacterium on Liquid Medium

Alternatively, Agrobacterium is prepared for transformation by growing in liquid medium. One day before infection, a 125-ml flask is prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate is suspended into the flasks and placed on an orbital shaker set at 200 rpm and incubated at 28° C. overnight. The Agrobacterium culture is centrifuged at 5000 rpm for 10 min. The supernatant is removed and the Agrobacterium infection medium (700A) with acetosyringone solution is added. The bacteria is resuspended by vortex and the optical density (550 nm) of the Agrobacterium suspension is adjusted to a reading of about 0.35 to 2.0.

### D. Maize Transformation

Ears of a maize (Zea mays L.) cultivar were surface-sterilized for 15-20 min in 20% (v/v) bleach (5.25% sodium hypochlorite) plus 1 drop of Tween 20 followed by 3 washes in sterile water. Immature embryos (IEs) were isolated from ears and were placed in 2 ml of the Agrobacterium infection medium (700A) with acetosyringone solution. The optimal size of the embryos varies based on the inbred, but for transformation with WUS2 and ZM-ODP2 a wide size range of immature embryo sizes was used. The Agrobacterium infection medium (810K) was drawn off and 1 ml of the Agrobacterium suspension was added to the embryos and the tube was vortexed for 5-10 sec. The microfuge tube was incubated for 5 min in the hood. The suspension of Agrobacterium and embryos were poured onto 7101 (or 562V) co-cultivation medium (see Table 3 and Table 4, respectively). Any embryos left in the tube were transferred to the plate using a sterile spatula. The Agrobacterium suspension was then drawn off and the embryos placed axis side down on the media. The plate was incubated in the dark at 21° C. for 1-3 days of co-cultivation and embryos were then transferred to resting medium (605B medium) without selection.

### Example 4: Production of a Doubled Haploid Inducer (DHI) Having Introduced Genetic Chromosome Doubling Factors

The present disclosure provides a maize maternal haploid induction system comprising maize lines containing a loss-of-function mutation at the MATRILINEAL (MATL) gene having a non-chemical, genetic mechanism to induce chromosome doubling of a one-celled haploid zygote. Disrupting the G1-S-G2-M (gap phase 1-synthesis phase-gap phase 2-mitosis) cell cycle to obtain a doubled haploid makes use of the transient zygotic state that precedes uniparental (male) chromosome elimination in maize maternal haploid induction. The present disclosure provides a method using haploid inducers comprising constructs integrated into the haploid inducer genome useful for disrupting the G1-S-G2-M cell cycle. These transformed haploid inducing lines are used as a parent in a cross between two plants. During pollination, expression of the genetic chromosome doubling factor from the paternal allele occurs before, during, or after fertilization (or a combination of the foregoing), providing chromosome doubling capabilities to an embryo sac cell (e.g., an egg cell). Such chromosome doubling activity in or near (or adjacent) the embryo sac cell, particularly the egg cell, stimulates embryogenesis of a cell containing paired maternal chromosomes. Any haploid inducer line capable of incorporating introduced genetic doubling factors is suitable for the methods described herein. Loss of function mtl1 allele is provided as an illustrative embodiment of an inducer line. In the case of maize, these inducer lines include those that are selected and/or derived from Stock 6, RWK, RWS, UH400, AX5707RS, and NP2222-matl.

Constructs described herein containing expression cassettes encoding genetic chromosome doubling factors are operably linked to a promoter that drives expression from the male genome, for example at the earliest stages post-fertilization. An exemplary promoter for this purpose is the proximal promoter of the Oryza sativa BABY BOOM 1 locus (BBM1; SEQ ID NO: 1), wherein zygotic expression of BBM1 is initially specific to the male allele. It is expected that promoter sequences of other BBM homologs are useful in the methods disclosed herein. For example, the promoter (SEQ ID NO: 9) and 5′ UTR (SEQ ID NO: 10) of the Zea mays BBM-2 locus may be useful in the methods of the present disclosure.

Alternatively, genetic chromosome doubling activity may be provided to the maternal haploid embryo by the male gamete prior to karyogamy. In this aspect, the genetic chromosome doubling activity is conferred by a protein expressed by the male gamete before, during, or after fertilization and before karyogamy. Regulatory elements comprising a promoter and 5′ UTR that are active in the male gamete are used in the methods of the present disclosure, including, but not limited to Zea mays regulatory elements from a profilin homolog2 locus (PRF2 PRO SEQ ID NO: 11 and 5′ UTR SEQ ID NO: 12), a pollen-specific protein SF3-like protein (SF3 PRO SEQ ID NO: 13 and 5′ UTR SEQ ID NO: 14), or a pectate lyase/amb allergen locus (PLAA PRO SEQ ID NO: 15 and 5′ UTR SEQ ID NO: 16).

Exemplary genetic chromosome doubling methods include, but are not limited to, proteins that allow endoreduplication of the maternal haploid genome, proteins that inhibit tubulin polymerization of the maternal haploid genome, and proteins that alter cell cycle regulation of the maternal haploid genome as described herein.

### A. Genetic Chromosome Doubling Using Endoreduplication

The method of the present disclosure allows obtaining a doubled maternal haploid by genetically disrupting the G1-S-G2-M cell cycle.

In an aspect, genetic chromosome doubling is achieved by allowing replication of the genome without cell division. An exemplary protein useful for stimulating genome duplication in the absence of completing the M-phase, characterized by completing cytokinesis and producing two daughter cells, is the mitotic inhibitor cell cycle switch 52 protein (CCS52). CCS52 blocks mitosis by degrading mitotic cyclins, leading to endocycles comprising G1-S-G2 phases. It is expected that the expression of the CCS52 protein is useful as a genetic chromosome doubling agent for controlling endoreduplication.

Such a CCS52 protein is a plant homologue of Anaphase-promoting complex activators (“APC”; also, called the “cyclosome”) that are involved in mitotic cyclin degradation, wherein the APC components comprise an E3 ubiquitin ligase that degrades targeted cell cycle proteins via the 26S proteasome. Overexpression of a WD-repeat-containing a yeast protein like CCS52 triggered mitotic cyclin degradation, cell division arrest, endoreduplication and cell enlargement in manner consistent with the methods of the present disclosure. Genetic chromosome doubling factors that are Zea mays homologs useful in the methods of the present disclosure are shown in Table 5.

TABLE 5

| | DNA SEQ | Protein SEQ |
|---|---|---|
| Endoreduplication factor | ID NO: | ID NO: |
| ZM-FIZZY-RELATED 2 | 18 | 24 |
| ZM-FIZZY-RELATED 2-like B | 19 | 25 |
| ZM-FIZZY-RELATED 2-like C | 20 | 26 |
| ZM-FIZZY-RELATED 2-like D | 21 | 27 |

In the methods of the present disclosure, it is expected that the maternal haploid embryo is provided genetic chromosome doubling activity from the male gamete of the double haploid inducer.

### B. Genetic Chromosome Doubling Using Tubulin Polymerization Destabilization

In a further aspect, a method for genetically disrupting the G1-S-G2-M cell cycle is provided in which tubulin polymerization is inhibited by expression of inhibitory proteins, thereby mimicking the anti-mitotic action of chemicals used as chromosome doubling agents.

Eukaryotic cells express several tubulin proteins, with α- and β-tubulin being the main components of microtubules. Microtubules for the mitotic spindle provides the framework for separating daughter chromatids during mitosis. Microtubules are comprised of 13 filaments and each filament is end-to-end heterodimers of α-β-tubulin that is laterally associated into a tube-like structure. Tubulin proteins are GTPases with an active site formed at the interface between subunits, using essential amino acid residues from both subunits. Moreover, GTPase activity only occurs when two or more subunits are associated, wherein the N-terminal domain of one subunit provides a nucleotide binding site and the C-terminal domain provides a “T7 loop” responsible for nucleotide hydrolysis.

Given that α-β-tubulin heterodimers join end-to-end, with distinct polarity (e.g. β-tubulin always present at the (+) end and α-tubulin at the (−) end), and GTP binding pockets between the two subunits, the methods of the present disclosure disrupt microtubule polymerization. For example, by expressing a mutated β-tubulin subunit, specifically in the N-terminal domain of the subunit providing nucleotide binding site, then nucleotide hydrolysis is prohibited. Thus, the mutated β-tubulin subunit acts as a “poisonous” subunit, or a dominant negative mutation, blocking polymerization on the (+) end of the polymer, thereby inhibiting filament polymerization. In yeast, expression of mutant α-tubulin TUB1-828: D252A/E255A (SEQ ID NO: 3, encoded by SEQ ID NO: 5) also inhibits spindle assembly, even when present only as a minor component of the total α-tubulin pool. Thus, expressing a mutated tubulin subunit causes dynamic instability of filaments causing an anti-mitotic mode of action useful as a genetic chromosome doubling agent.

In an aspect, missense mutations within a conserved site, such as the tubulin conserved site [SAG]-G-G-T-G-[SA]-G (Prosite accession number PS00227), wherein a polynucleotide is used encoding missense mutations, for example H-A-A-H-R-Q-A residues substituted into the PS00227 site, herein called “β-tubulin-PS00227minus” (polynucleotide SEQ ID NO: 22; encoding SEQ ID NO: 28) are expected to provide genetic chromosome doubling activity to the maternal haploid embryo from the male gamete of the double haploid inducer.

### C. Genetic Chromosome Doubling Using Altered Cell Cycle Regulation

In a further aspect, the genetic chromosome doubling agent is a cyclin gene capable of altering cell cycle regulation before, during, or after fertilization. Genetic chromosome doubling of a parthenogenic haploid embryo has been shown by providing to an egg cell the activity of a cyclin gene, dpzm07g031470.1.1, herein called ZM-CYCD2 (SEQ ID NO: 17) encoding a cyclin delta-2-like protein (SEQ ID NO: 23). The activity of this cyclin gene during female gametogenesis provided chromosome doubling activity of a haploid embryo in vivo, thereby creating a diploidized (2n) embryo having only maternal chromosomes.

It is expected that these genes will be useful in the methods of the present disclosure in providing genetic chromosome doubling activity to the maternal haploid embryo from the male gamete of the double haploid inducer.

### D. Method for Obtaining a Doubled Haploid Inducer

The methods disclosed herein use a plasmid having an expression cassette with a polynucleotide comprising genetic chromosome doubling factors as described above. Plasmids useful for the current method are described in Tables 2 and 6.

TABLE 6

| SEQ | | Genetic Chromosome Doubling Expression Cassette | | |
|---|---|---|---|---|
| ID | | Regulatory | Doubling | Doubling |
| NO: | Plasmid | Element | Factor | Mechanism |
| 29 | RV044054 | ZM-BBM2 | ZM-CYCD2 | cell cycle regulation |
| 30 | RX000002 | | ZM-FIZZY-RELATED 2-like | endoreduplication |
| 31 | RX000003 | | ZM-FIZZY-RELATED 2-like B | |
| 32 | RX000004 | | ZM-FIZZY-RELATED 2-like C | |
| 33 | RX000005 | | ZM-FIZZY-RELATED 2-like D | |
| 34 | RX000006 | | tubulin-PS00227minus | microtubule destabilization |
| 35 | RV042024 | ZM-PRF2 | ZM-CYCD2 | cell cycle regulation |
| 36 | RX000007 | | ZM-FIZZY-RELATED 2-like | endoreduplication |
| 37 | RX000008 | | ZM-FIZZY-RELATED 2-like B | |
| 38 | RX000009 | | ZM-FIZZY-RELATED 2-like C | |
| 39 | RX000010 | | ZM-FIZZY-RELATED 2-like D | |
| 40 | RX000011 | | tubulin-PS00227minus | microtubule destabilization |
| 41 | RV042025 | ZM-SF3 | ZM-CYCD2 | cell cycle regulation |
| 42 | RX000012 | | ZM-FIZZY-RELATED 2-like | endoreduplication |
| 43 | RX000013 | | ZM-FIZZY-RELATED 2-like B | |
| 44 | RX000014 | | ZM-FIZZY-RELATED 2-like C | |
| 45 | RX000015 | | ZM-FIZZY-RELATED 2-like D | |
| 46 | RX000016 | | tubulin-PS00227minus | microtubule destabilization |
| 47 | RV042026 | ZM-PLAA | ZM-CYCD2 | cell cycle regulation |
| 48 | RX000017 | | ZM-FIZZY-RELATED 2-like | endoreduplication |
| 49 | RX000018 | | ZM-FIZZY-RELATED 2-like B | |
| 50 | RX000019 | | ZM-FIZZY-RELATED 2-like C | |
| 51 | RX000020 | | ZM-FIZZY-RELATED 2-like D | |
| 52 | RX000021 | | tubulin-PS00227minus | microtubule destabilization |
| 67 | RV044484 | OS-BBM1 | ZM-CYCD2 | cell cycle regulation |

For these experiments, a maize plant selected and/or derived from Stock 6, RWK, RWS, UH400, AX5707RS, and NP2222-matl, or any of the several other haploid inducer lines is transformed. It is also expected that a maize plant containing a mutation at the MATRILINEAL (MATL) gene is used in the methods of the present disclosure. Preferentially, the selected maize plant has a paternal morphological marker gene as described in U.S. Pat. No. 8,859,846 B2, incorporated by reference herein in its entirety. Here, a haploid detection method is used comprising the detection of a paternal morphological marker. A paternal morphological marker may comprise a fluorescent reporter expression construct, such as a green, yellow, or red fluorescent reporter gene, that allows the fluorescence detection in the embryos at the early developmental stage and/or an allele of the anthocyanin genes, such as the R1-scm allele, expressing in embryos at the early developmental stage. Such marker genes allow identifying diploid and haploid embryos based on the presence or absence of the paternal marker gene products, respectively.

Transformed doubled haploid inducer plants were created using the methods described in Example 3, using plasmids RV044054 (SEQ ID NO: 29), RV042024 (SEQ ID NO: 35), RV042025 (SEQ ID NO: 41), RV042026 (SEQ ID NO: 47), and RV044484 (SEQ ID NO: 67), described in Tables 2 and 6. Regenerated T₀ plants are analyzed by qPCR to identify single copy, hemizygous plants containing each plasmid. Propagating such plants results in obtaining and using single copy, homozygous plants for haploid induction crosses. In addition, it is expected that the remaining plasmids listed in Table 6 will be useful in creating transformed doubled haploid inducer plants as described in Example 3 for use in the methods disclosed herein.

Any such doubled inducer plants, or progeny thereof, are grown and used as a pollen donor to fertilize a donor ear of a female plant comprising a haploid induction cross. The ears of the female parent plants are shoot-bagged before silk emergence to avoid any foreign pollen contamination. The silks of the ears on the plants of the female parent plants are pollinated with viable pollen grains collected from the anthers of the doubled haploid inducer.

In an aspect, at approximately 9-21 days after pollination, ideally at 18 days, the immature ears are harvested. The ears are surface sterilized in 30% Clorox bleach plus 0.5% detergent for 20 minutes and rinsed two times with sterile water. The haploid embryos are isolated based on the identification of the visible marker gene in the inducer lines. For example, use of an inducer containing a fluorescent reporter gene or an anthocyanin biosynthesis gene operably linked to a promoter allowing gene expression in the embryos at the early developmental stage.

Alternatively, mature ears are harvested, seed is collected, and seed containing haploid or diploid embryos are identified based on the absence and presence of a paternal morphological marker gene, respectively.

It is expected the methods of the present disclosure using a doubled haploid inducer will eliminate the need to perform a chromosome doubling treatment. It is also expected that both the quantity and quality of the plants per DH (doubled haploid) breeding population will be improved. It is also expected that the doubled haploid inducer method described herein will improve both human safety and environmental health concerns in comparison to current methods that use certain chemical chromosome doubling treatments.

### Example 5. Development, Maintenance and Utility of a Doubled Haploid Inducer (DTII) System

The method of the current example described the maintenance and use of a doubled haploid inducer. Genetic chromosome doubling methods are described in Example 4 and related concepts are further described here as a method comprising a doubled haploid inducer system.

In one aspect, the current method describes transformation plasmids with an inactive genetic chromosome doubling trait during plant transformation, regeneration, and sporophytic growth that becomes active in the male gamete. In this manner, it is expected that the doubled haploid inducer is propagated and maintained as a diploid plant in most cells, except for a male gamete cell. In this manner the male gamete confers subsequent chromosome doubling activity to a maternal egg cell. Thus, in a second aspect, the example described a method for obtaining a maternal haploid plant that has been diploidized in vivo, also known as a “maternal di-haploid” plant, here obtained without use of a chemical chromosome doubling agent.

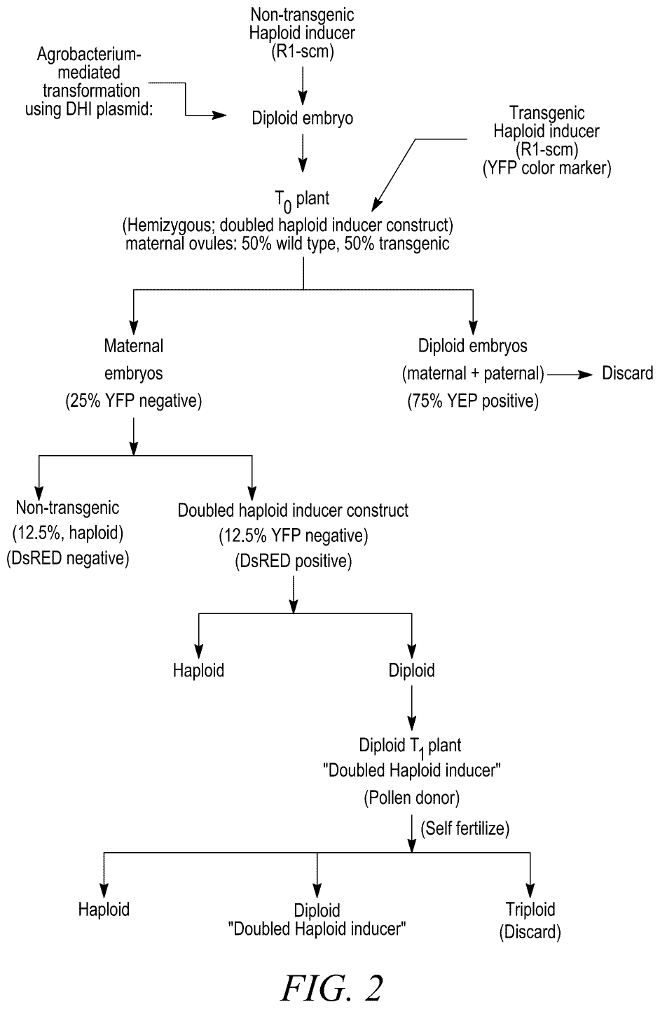

Here the method for obtaining a stably transformed plant is described (see FIG. 2). Briefly, immature diploid embryos of a haploid inducer lacking a fluorescent protein marker are transformed using Agrobacterium strain LBA4404 THY- (See U.S. Pat. No. 8,334,429 incorporated herein by reference in its entirety). Transformation is performed using a mixture of Agrobacteria, as described in US Patent Application Publication Number 2021/0062203 incorporated herein by reference in its entirety. An Agrobacterium strain having plasmid RV020636 (SEQ ID NO: 56) is used to obtain transgenic plants with a single-copy of an integrated T-DNA from a “doubled haploid inducer” (DHI) plasmid.

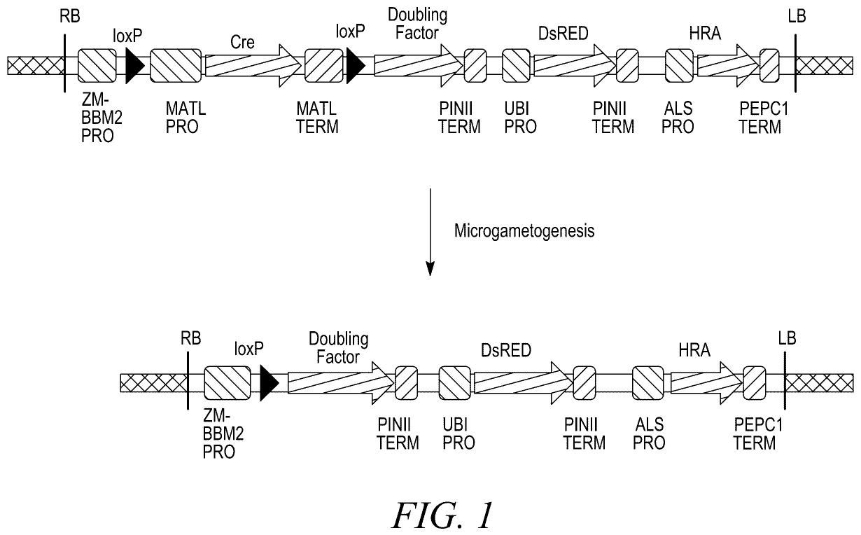

An exemplary DHI plasmid (SEQ ID NO: 55; see FIG. 1) described herein contains a polynucleotide with a first expression cassette comprising the ZM-CYCD2 (SEQ ID NO: 17) with chromosome doubling activity that is operably linked to the Zea mays BBM2 promoter and 5′ UTR (SEQ ID NO: 9-10, respectively). The first expression cassette contains a second, intervening expression cassette flanked by loxP sites. This second, intervening expression cassette encodes a Cre recombinase protein operably linked to the promoter and terminator of the Zea mays MATRILINEAL (MATL) gene (SEQ ID NO: 53 and SEQ ID NO: 54, respectively). It is expected that expression of Cre will occur during microgametogenesis and will excise the second, intervening expression cassette, thereby allowing expression of the genetic chromosome doubling factor upon delivery of the paternal chromatin to the egg cell. A doubled haploid inducer plasmid using any of the genetic chromosome doubling factors described in Example 4, either in substitution of ZM-CYCD2 or in any other combination are useful in the methods disclosed herein. The DHI plasmid also contains expression cassettes encoding a reporter marker gene, such as a red fluorescent protein, and a selectable marker gene. In this experiment, a mixture comprising 90% of the Agrobacterium strain having the DHI plasmid and 10% of the plasmid RV020636 (v/v) (SEQ ID NO: 56) is used for transformation.

Following co-infection of a diploid embryo, somatic embryogenesis is activated in response to RV020636 (SEQ ID NO: 56) activity and somatic embryos are cultured as described in Example 3. After approximately 6-10 days any proliferating tissue and somatic embryos are dissected and sub-cultured, wherein each portion of dissected tissue is transferred to maturation medium (289Q) for in vitro culture at 26-28° C. under dark conditions. After approximately 6-10 days the sub-cultured tissues are transferred to a light culture room at 26° C. until healthy plantlets with good roots develop. Approximately 7-14 days later, plantlets are transferred to flats containing potting soil and grown for 1 week in a growth chamber, subsequently grown an additional 1-2 weeks in the greenhouse, and then transplanted to soil in pots and grown under greenhouse conditions.

To identify T₀ plants having a stably integrated T-DNA provided by the DHI plasmid and lacking the T-DNA of plasmid RV020636 (SEQ ID NO: 56), leaf tissue is sampled per plant and is evaluated using PCR diagnostic methods. Plants lacking the RV020636 plasmid sequence (SEQ ID NO: 56) that are diploid and single copy for the DHI plasmid are selected, wherein each plant comprises a unique event. Selected T₀ plants are grown to maturity.

The ears of the selected T₀ plants are shoot-bagged before silk emergence to avoid any foreign pollen contamination. The silks of the ears on the plants of the female parent plants are pollinated with viable pollen grains collected from a doubled haploid inducer. Preferentially, the haploid inducer possesses a unique morphological trait, such as a transgene encoding a fluorescent protein reporter gene that differs from the reporter gene provided by the DHI plasmid, for example a transgene encoding a ZsYellow fluorescent protein reporter.

At approximately 9-14 days after pollination, the immature ears are harvested. The ears are surface sterilized in 30% bleach plus 0.5% Micro detergent for 20 minutes and rinsed two times with sterile water. T₁ embryos are dissected and cultured as described in EXAMPLE 3 and, here with the inclusion of a chromosome doubling step, such as contacting a plant cell with colchicine at a concentration of 0.1 to 1.0 g/ml for a period of 24 hours before transfer to a resting medium (605J) that is lacking a chromosome doubling treatment. Alternatively, the chromosome doubling step is performed at a later time, such as using root soaking methods.

After 24 hours, T₁ embryos are scored the absence/presence of the paternal morphological trait, such as expression of a ZsYellow fluorescent protein, wherein haploids and diploid embryos are detected based on the absence or presence of said trait, respectively. In the methods disclosed herein, it is expected that haploid induction cross will create a proportion of maternal haploid embryos with half expected to be non-transgenic and half expected to have inherited the DHI construct. Identification of the transgenic haploid embryos will be characterized by the absence of the paternal morphological trait and the presence of the DsRED trait (see FIGS. 1 and 2). Non-transgenic haploid embryos will lack both the paternal morphological trait and the presence of the DsRED trait and are discarded.

T₁ plants having the stably integrated T-DNA provided by the DHI plasmid and lacking in the T-DNA of plasmid RV020636 (SEQ ID NO: 56) that have been diploidized are potted to soil and grown to maturity. Such plants are expected to be homozygous for the DHI trait and 100% of the pollen grains are expected to have the DHI construct that becomes active during microgametogenesis (see FIG. 1). Briefly, the Cre recombinase expression cassette that that is operably linked to the MATL promoter will express in the pollen grain, thereby allows the expression of the doubling factor to be operably linked to the ZM-BBM2 promoter. Other expression cassettes encoding other genetic chromosome doubling factors are useful in the methods disclosed herein, for example as described in Example 4, as well as operably linking said expression cassettes to other useful promoters, for example as described in Example 4.

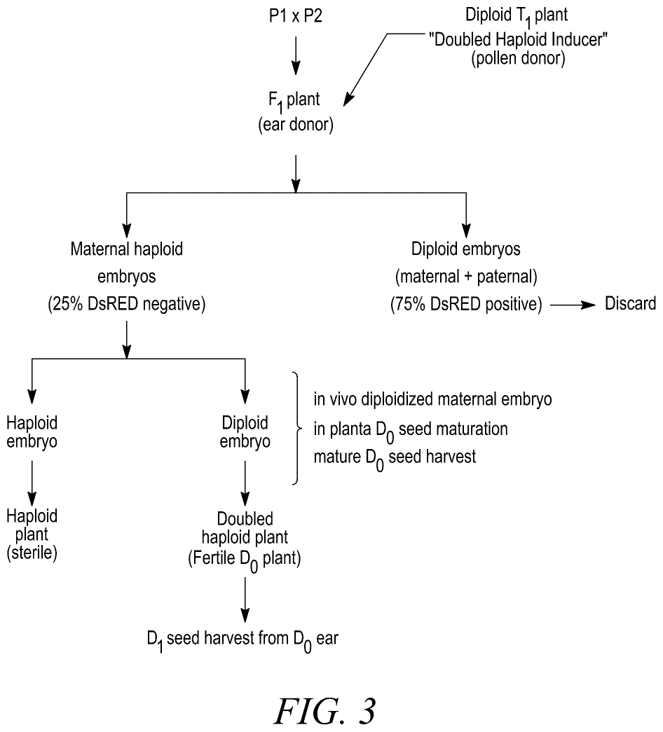

It is expected that maintaining a selected T₁ plant is performed by self-fertilization and results in producing the following outcomes: a haploid embryo (paternal genome elimination without doubling of maternal chromosome), a diploid embryo (paternal genome elimination and doubling of maternal chromosome; the desired outcome to obtain a DHI plant), or a triploid embryo (inherited paternal genome (1n) and a doubled maternal genome (2n)) (see FIG. 2). It is expected that a diploid T1 plant having 2 copies of the DHI construct is confirmed using copy number analysis methods that identify and select such a plant, or progeny thereof, as doubled haploid inducers, as shown in FIG. 3.

In the current method, utility of a DHI line is shown in FIG. 3, wherein pollen from a DHI plant is collected and used to fertilize a donor ear, for example the ear of a F1 hybrid resulting from a biparental cross that is obtained and processed using methods described above. It is expected the DHI construct will become active as described above (see FIG. 1), thereby producing an embryo either with or without the paternal genome. Embryos with the paternal genome are expected to be detected using the paternal morphological marker gene, for example DsRED activity and such diploid embryos are discarded. Embryos lacking the paternal genome are detected based on the absence of the paternal morphological marker gene, for example an embryo that does not express a DsRED protein. Such maternal embryos that are lacking the paternal marker are selected and transferred to a growth medium to obtain a plantlet.

It is expected that a diploidized maternal embryo is obtained in response to the genetic chromosome doubling activity provided by the paternal genome during or after fertilization using the methods disclosed herein. It is expected that such activity provided to a haploid maternal egg cell creates doubled haploid embryos in vivo. It is expected that such embryos do not require tissue culturing methods using a chemical doubling agent (see FIG. 3).

### Example 6: Automated Methods to Identify In Vivo Diploidized Maternal Embryos

As described in Example 5, while it is an expected goal and purpose of the methods disclosed herein to obtain in vivo doubled haploid embryos, the result of Example 5 may produce a population of maternal embryos as a combination of in vivo doubled haploid embryos and maternal haploids. Thus, a method to discern haploids and diploids is useful in the methods of the present disclosure. This example describes methods for this purpose.

For example, a population of maternal embryos is analyzed using an apparatus for preparing a plant tissue, to hold a tissue, for transferring plant tissue, for culturing plant tissue, and/or for subjecting the tissue to a force to divide the tissue into separate segments. It is expected such steps use more than one apparatus.

It is expected that the above automation steps also comprise integrating analytical capabilities including, but not limited to, use of different sensors and image capture systems. For example, an apparatus is used for acquiring an image, such as visual, hyperspectral, thermal, or fluorescence imaging apparatus. In another aspect, an apparatus is used for measuring, capturing, and analyzing qualitative and quantitative traits, such as biomass, shape, thickness, volume, growth rate, and/or morphological characteristics. In an aspect, the apparatus is also used for measuring and analyzing other parameters, such as light intensity, light duration, water and nutrient uptake, evaporation and transpiration, and/or quantitative measurement of the complete set of ions or changes in ion production under varied external stimuli.

In another aspect, a method comprising the analysis of a sampled tissue that is collected using at least one apparatus is integrated with methods for performing a ploidy analysis, for example methods using a flow cytometry. Such methods include, but are not limited to, collecting tissue, isolating intact nuclei, adding various buffer, reagents, and dyes to isolated nuclei, and automated transfer and loading of the sample into a flow cytometer.

The acquired images and data are for data analysis and interpretation; no effort is made here to describe all methods of acquisitions pertinent to the current example. Such methods include performing feature extraction for classification purposes, wherein such classification is used for predictive model generation. Such predictive modelling is a computer implemented model encompassing a variety of statistical techniques from data mining, automated feature extraction, and machine learning.

It is also expected that such data analysis comprises linking genotypic data with captured phenotypic data, including methods measuring biomolecules from a tissue before, during, or after such automation methods are performed.

It is expected that the results of the automated methods described herein are linked with genotypic data, for example to enable predicting phenotypic performance using a biological model based on genomic data of a characterized plant, tissue, or plant cell that was generated and/or treated using the methods disclosed herein.

It is expected that the methods disclosed herein improve the capability for identifying and selecting diploidized maternal embryos from haploid maternal embryos, including, but not limited to, aspects for improving the regeneration frequency, improved transplanting success, and ultimately improvements for the reproductive success of the diploidized plants produced using such automated treatment, handling, and phenotyping methods. It is expected that productivity gains are achieved when populations are enriched for diploidized maternal embryos that result in fertile plants.

In another aspect, it is expected that the results of linking genotypic data with the acquired phenotypic data will enable improving predicting phenotypic performance using a biological model based on genomic data of a characterized plant, tissue, or plant cell treated.

It is furthermore expected that the methods disclosed herein improve plant breeding outcomes, for example when such automated treatment, handling, and phenotyping methods that co-integrate the above data analysis and interpretation with methods of selecting such doubled haploid plants based on a genomic estimated breeding value are used.

It is expected these methods provide an improved productivity of a non-random, structured breeding population resulting in cost-effectively providing a population with the number of individuals required having some specified quantity and/or quality of interest. Thus, in comparison to having the same probability in the idealized population, such as the effective population size required when provided a random population, the methods disclosed herein improve the relative rate of genetic gain while using relatively fewer resources.

### Example 7: Alternative Maintenance Methods of a Doubled Haploid Inducer (DHI)

Doubled haploid inducers (DHI) as described in Examples 4 need a means of preventing the doubling process from occurring during seed increases (self-pollination) of the DHI. Moreover, most transformation processes involve an embryogenesis step, which may activate constructs as described in Example 4 when driven by the BABY BOOM 1 promoter. Such maintainer constructs generally have 3 components: (i) prevention of transmission through the pollen, (ii) a seed color marker gene to identify seeds which contain the maintainer construct, and (iii) a counteracting factor which negates the effect or phenotype of a trait, either present on another construct or in the natural plant genome.

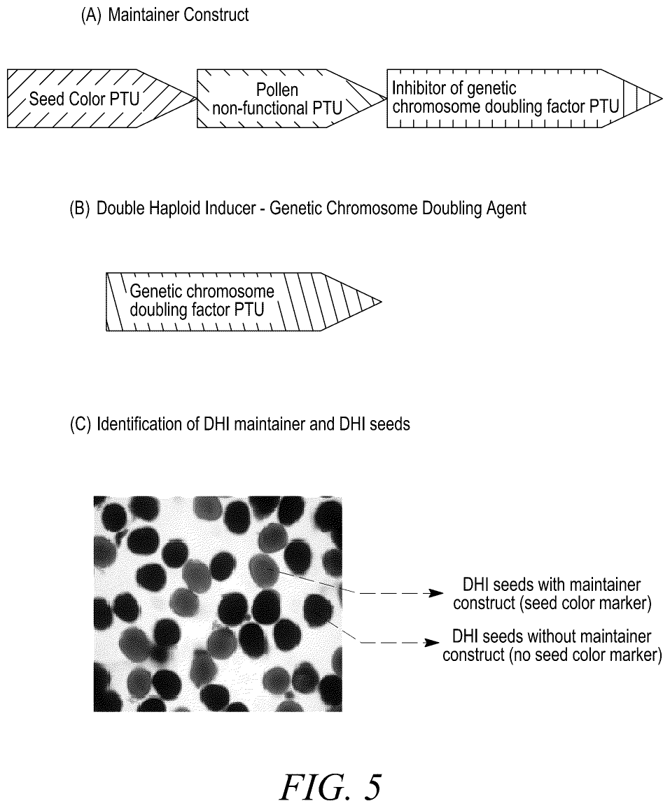

An exemplary general maintainer construct for maintaining the DHI line is shown in FIG. 5. The maintainer construct is designed to allow seed production of the doubled haploid inducer (DHI) line without an increase in ploidy caused by the genetic chromosome doubling factor containing plant transcription unit (PTU). In this example, the maintainer construct includes three components: a seed color PTU to identify the maintainer construct-containing seeds resulting from the self-pollination; a pollen non-functional PTU to prevent transmission of the maintainer construct through the pollen (FIG. 5A); and an inhibitor of the genetic chromosome doubling factor PTU to keep the DHI in the diploid state. All functional pollen will contain only the genetic chromosome doubling factor PTU (FIG. 5B) and will induce doubled haploids (DH) in the induction cross. The maintainer construct will transmit through female gametes and will be present in the heterozygous state in ˜50% of DHI self-pollinated seeds (FIG. 5C). Such seeds are used for further seed increases and/or DH induction.

An example of the first component is a maize alpha-amylase gene in which the native signal peptide was replaced by the amyloplast-targeting signal peptide from the maize brittle-1 gene driven by the late pollen-specific promoter from a maize polygalacturonase gene, PG47 (SEQ ID NO: 6; see Wu et al. 2016. Plant Biotechnology Journal 14:1046-1054). An example of the second component is a polynucleotide encoding a DsRed2 protein coding sequence (SEQ ID NO: 8) driven by a promoter from a barley lipid transfer protein (LTP2) gene, which is preferentially expressed in the endosperm (SEQ ID NO: 7; see Wu et al. 2016. Plant Biotechnology Journal 14:1046-1054). The third component is designed to inhibit the transcription, translation, and/or activity of the genetic mechanisms that induce chromosome doubling described in Example 4.

Artificial microRNA (amiRNA)-mediated gene silencing represents one of such techniques. amiRNA technology exploits the microRNA (miRNA) biogenesis pathway to produce artificially designed small RNAs using the miRNA gene backbone. It generates a single type of small RNA population all with the same selective nucleic acid sequence, usually 21 nucleotides (nt) in length, providing a feasible method for either silencing an individual gene or simultaneously silencing closely related gene isoforms. Codon usage differences are used to specifically target the genetic mechanism present in the construct without affecting the expression of the endogenous form of said genetic mechanism.

Transcriptional repressors are proteins that bind to specific sites on DNA and prevent transcription of nearby genes, and represent another technique to inhibit the transcription, translation or activity of the genetic mechanisms that induce chromosome doubling. The promoter driving the genetic mechanism for chromosome doubling are modified to contain repressor binding sites. The repressor protein will not be present in the active pollen of a plant containing the maintainer construct, allowing the genetic mechanism of chromosome doubling to activate in the zygote following fertilization.

An exemplary method for inhibiting chromosome doubling activity is the use of repressor proteins. In the absence of the cognate ligand, a transcriptional repressor protein acts by binding to specific DNA sequences in operator regions, or “operators”. For example, transcriptional repressors belonging to the tetracycline repressor (TetR) family of bacterial regulatory proteins are one such class of transcriptional repressors of interest to the current method. In Escherichia coli, in the absence of tetracycline, TetR binds to the tetracycline operator (tetO) sequence and such binding affinity to TetR/operator sequences represses operon gene expression. In the presence of tetracycline, the conformation of a TetR-tetracycline complexes is changed, TetR/operator binding affinity is reduced, the TetR complex dissociates from the tetracycline operator, and gene expression of the operon is activated.

Here, a method for conditional transcriptional repression is described using the TetR/operator system to inhibit the transcription, translation or activity of the genetic mechanisms that induce chromosome doubling.

In one embodiment, the method of this Example 7 a plant with a stably incorporated chromosome doubling expression cassette containing a polynucleotide encoding a genetic chromosome doubling factor that is operably linked to an embryogenic promoter, such as the O. sativa BABY BOOM 1 promoter (SEQ ID NO: 1) having at least one tetracycline operator sequence, here called “TET OP1 (PHI)” (SEQ ID NO: 57) is disclosed. Genetic doubling factors include, but are not limited to, the fizzy related sequences described in Table 5; mutated tubulin coding sequences, such as mutant α-tubulin TUB1-828: D252A/E255A (SEQ ID NO: 3, encoded by SEQ ID NO: 5) or tubulin-PS00227minus (SEQ ID NO: 22); and a cyclin gene, such as ZM-CYCD2 (SEQ ID NO: 17).

To repress the above chromosome doubling expression cassette, an exemplary method of the current example also comprises the stably transformed plant having a conditional TetR expression cassette containing a polynucleotide encoding a tetracycline repressor that is operably linked to the Cauliflower mosaic virus 35S promoter (SEQ ID NO: 58). For example, a tetracycline repressor used here is a Zea mays codon optimized synthetic tetracycline repressor protein (polynucleotide SEQ ID NO: 59, encoding SEQ ID NO: 60). In this example, the repressor protein will not be present in the active pollen of a plant containing the maintainer construct yet will be expressed in other cell types. For this reason, the Cauliflower mosaic virus 35S promoter is a useful regulatory element given its activity in sporophytic cells and lack of activity in maize pollen (see Table 3 of Koziel, et al. 1993. Nat Biotechnol 11:194-200; https://doi.org/10.1038/nbt0293-194). Thus, it is expected the TetR protein will be active during the sporophytic growth, during megagametogenesis, yet not during microgamatogenesis.

It is expected that the combination of these two expression cassettes (a chromosome doubling expression cassette and a conditional TetR expression cassette) comprise a method for providing a counteracting factor which negates the effect or phenotype of the chromosome doubling trait allowing the genetic mechanism of chromosome doubling to activate in the zygote following fertilization.

In this manner, it is expected that a maintainer construct comprising (i) prevention of transmission through the pollen, (ii) a seed color marker gene to identify seeds which contain the maintainer construct, and (iii) a counteracting factor which negates the effect or phenotype of a trait are integrated components for methods to maintain a doubled haploid inducer.

### Example 8: Genetic Chromosome Doubling Using Alternative Cyclin Genes

Methods for in vivo genetic chromosome doubling using a transformed haploid inducer line are useful in the methods of the present disclosure. The method of Example 4 describes in vivo genetic chromosome doubling methods, for example altering cell cycle regulation of a female gamete, such as egg cell. Of particular interest is the method described in Example 4 C., wherein a cyclin gene confers genetic chromosome doubling activity of a haploid embryo in vivo. Additional cell cycle regulation genes are of interest for use in the methods of the present disclosure.

This Example 8 describes methods using alternative cyclin genes for the genetic chromosome doubling trait in combination with the haploid induction provided by the paternal genome of a haploid inducer line, such as Stock 6 or any derivative thereof.

The methods of the present disclosure simplify doubled haploid production logistics, reduce the cost of materials, reduce the demand for labor, improve safety by reducing lab usage of chemical doubling agents, and mitigate the negative impacts of attrition that occur despite obtaining a sample of embryos from a donor ear derived from a haploid induction cross.

Genetic chromosome doubling is performed using a cyclin protein family member as an alternative to the Dz470 protein. Preferentially, the cyclin protein family member is a cyclin capable of linking growth and cell cycle control, such as a D-type cyclin. For example, D-type cyclins that have homology to Dz470, a cyclin delta-2 protein.

Using methods described in Example 4, it is expected that plasmids are created and used for transformation into a plant as described in Example 3, using a DNA polynucleotide encoding a cyclin family member shown in Table 7.

TABLE 7

| | | | | DNA | PRT |
|---|---|---|---|---|---|
| | | | Hit | SEQ | SEQ |
| Homolog ID | Organism | Relation | Description | ID NO: | ID NO: |
| At2g22490.1 | Arabidopsis | ortholog | CYCD2; 1 | 68 | 86 |
| | thaliana | | | | |
| At5g65420.1 | Arabidopsis | family | CYCD4; 1 | 69 | 87 |
| | thaliana | | | | |
| At5g10440.1 | Arabidopsis | family | CYCD4; 2 | 70 | 88 |
| | thaliana | | | | |
| Glyma14g35850.1 | Glycine | ortholog | D-type cyclin | 71 | 89 |
| | max | | | | |
| Glyma02g37560.1 | Glycine | family | Cyclin | 72 | 90 |
| | max | | | | |
| Glyma01g03030.1 | Glycine | family | Cyclin | 73 | 91 |
| | max | | | | |
| Glyma18g21730.1 | Glycine | family | Cyclin | 74 | 92 |
| | max | | | | |
| Os09g29100.1 | Oryza | candidate | cDNA putative, | 75 | 93 |
| | sativa | | expressed | | |
| Os08g37390.1 | Oryza | family | cDNA putative, | 76 | 94 |
| | sativa | | expressed | | |
| Os07g42860.1 | Oryza | family | cDNA putative, | 77 | 95 |
| | sativa | | expressed | | |
| Sb02g027020.1 | Sorghum | ortholog | D-type cyclin | 78 | 96 |
| | bicolor | | | | |
| Sb07g023350.1 | Sorghum | family | D-type cyclin | 79 | 97 |
| | bicolor | | | | |
| Sb02g039550.1 | Sorghum | family | D-type cyclin | 80 | 98 |
| | bicolor | | | | |
| dpzm04g027750.1.1 | Zea mays | family | Cyclin delta-2 | 81 | 99 |
| dpzm04g051480.1.1 | Zea mays | family | Cyclin, C- | 82 | 100 |
| | | | terminal domain | | |
| | | | protein | | |
| dpzm05g068750.1.1 | Zea mays | family | Cyclin, C- | 83 | 101 |
| | | | terminal domain | | |
| | | | protein | | |
| dpzm01g064180.1.1 | Zea mays | family | D-type cyclin | 84 | 102 |
| dpzm02g059300.1.1 | Zea mays | family | Cyclin | 85 | 103 |

It is expected that providing the simultaneous activity of the haploid induction characteristic of a haploid inducer line combined with the genetic chromosome doubling activity provided by an alternative cyclin gene family member is used to obtain in vivo diploidized maternal embryos.

The benefits of providing the simultaneous activity of the haploid induction characteristic of a haploid inducer line combined with the genetic chromosome doubling activity are numerous. These benefits include, but are not limited to, the elimination of 1) performing haploid induction crosses that require planting both a donor plant and a haploid inducer plant, 2) monitoring embryo development after performing the haploid induction cross, 3) harvesting the donor ear of said haploid induction cross in a timely manner based on embryo development, 4) isolating embryos from said donor ear (often a labor-intensive and tedious process), 5) contacting isolated embryos with a chemical chromosome doubling agent (a process that poses safety and health concerns of exposure to mammalian cells), 6) removing treated embryos from contact with said chemical chromosome doubling agent, 7) identifying and sorting haploid from diploid embryos, 8) transferring selected haploid embryos for continued in vitro tissue culture propagation, 9) regenerating a plantlet from said tissue culture steps, 10) hardening the plantlet, 11) transplanting said hardened plantlet, and 12) the negative impacts that occur at each step that result in impaired development, and more importantly impaired fertility, of said doubled haploid plant. The methods disclosed herein positively impact logistics resulting in cost savings and provide productivity gains for breeding programs using doubled haploid technologies through reduced attrition of haploid embryos throughout the process.

### Example 9: Genetic Chromosome Doubling Using Wide Hybridization

Wide hybridization comprises the exchange or modification of the genes due to crossing between species from distant gene pools. Crossing between individuals of different species or genera, herein called interspecific and intergeneric hybrids, respectively, provides a way to combine diverged genomes into one nucleus. Wide hybridization crosses useful in the methods disclosed herein include, but are not limited, to methods for obtaining interspecific or intergeneric hybrids.

Progeny of wide hybridization crosses wherein one parent of the wide hybridization cross is transformed with a construct containing a heterologous construct encoding a genetic chromosome doubling factor are useful in the methods disclosed herein. The parent having a genetic chromosome doubling trait is used as a pollen donor with the pollen being used to fertilize the maternal gametophyte of the second parent. It is expected that the genetic chromosome doubling activity of the first parent is provided to the maternal gametophyte of the second parent. It is expected that the maternal gametophyte of the second parent will be a haploid (1n) cell that becomes an in vivo diploidized (2n) cell comprising two sets of maternal chromosomes. Such an in vivo diploidized (2n) cell is used to obtain an embryo that is used to obtain a doubled haploid plant. The progeny obtained by self-fertilization of such a doubled haploid plant are used in plant breeding.

It is expected that the genome of the first parent having a genetic chromosome doubling trait will be eliminated from the maternal cell, for example, by genome elimination before, during or after fertilization. Wide crosses using cultivated maize (Zea mays) as the second parent will provide a maternal gametophtye that is fertilized with pollen obtained from a first parent. Pollen is obtained from any plant that is not normally sexually compatible Z. mays including, but not limited to, pollen obtained from the Poaceae Family, preferentially from a Glade including Panicoideae, Arundinoideae, Chloridoideae, Micrairoideae, Aristidoideae, and Danthonioideae subfamilies, and more preferentially, from the Andropogoneae subfamily, also known as a “supertribe”, comprising the Paspaleae, Andropogoneae, and Arundinelleae tribes. Of particular interest is the Tripsacinae subtribe of the Andropogoneae, wherein any species of the Genus Elionurus, Oxyrhachis, Rhytachne, Tripsacum, Urelytrum, Vossia, or Zea can be used as a pollen donor for performing wide hybridization.

It is expected that a pollen donor that is stably transformed with a heterologous construct containing a polynucleotide conferring genetic chromosome doubling activity is obtained. For example, a polynucleotide encoding an expression cassette as described in Example 4.

It is expected the first parent used as the pollen donor provides genetic chromosome doubling activity to the maternal gametophyte of the second parent. As an outcome, it is expected that the haploid (1n) maternal gametophyte contacted with the genetic chromosome doubling activity provides an in vivo diploidized (2n) embryo comprising two sets of maternal chromosomes. Such an in vivo diploidized (2n) embryo is used to obtain a doubled haploid plant, here achieved without treating the maternal cell with a chemical chromosome doubling agent. It is expected that the diploidized (2n) embryo produces a doubled haploid plant, and the progeny thereof, in a manner that is useful for plant breeding.

That which is claimed:

1. A method of producing a doubled haploid plant, comprising:
a) providing a first plant, wherein the first plant comprises at least one introduced genetic chromosome doubling agent;
b) crossing the first plant with a second plant, wherein the at least one introduced genetic chromosome doubling agent of the first plant induces chromosome doubling of a fertilized egg cell of the second plant in the absence of an exogenously applied chemical or biochemical chromosome doubling agent;
c) obtaining a diploidized embryo comprising a pair of chromosomes inherited from the second plant and not comprising the introduced genetic chromosome doubling agent; and
d) regenerating a diploid plant from the diploidized embryo.

2. The method of claim 1, wherein the first plant is a haploid inducer.

3. The method of claim 2, wherein the haploid inducer comprises a loss-of-function mutation in a patatin-like phospholipase A2α gene.

4. The method of claim 3, wherein the loss-of-function mutation in the patatin-like phospholipase A2α gene is the MATRILINEAL (MATL) gene.

5. The method of claim 2, wherein the first plant expresses a marker gene.

6. The method of claim 5, wherein the marker gene is selected from a selectable marker, a reporter gene, a visible endogenous morphological marker, and combinations thereof.

7. The method of claim 6 wherein the selectable marker is selected from the group consisting of GUS, PMI, PAT, and combinations thereof.

8. The method of claim 6, wherein the reporter gene is selected from the group consisting of GFP, RFP, CFP, and combinations thereof.

9. The method of claim 6, wherein the visible endogenous morphological marker is selected from the group consisting of B1, R-nj, R1-scm, anthocyanin pigments, and combinations thereof.

10. The method of claim 1, wherein the genetic chromosome doubling agent is a genetic chromosome doubling polypeptide.

11. The method of claim 10, wherein the genetic chromosome doubling polypeptide is selected from the group consisting of:
a) a polypeptide allowing replication of a genome without cell division;
b) a polypeptide destabilizing tubulin polymerization;
c) a polypeptide altering cell cycle regulation; and
d) combinations of the foregoing.

12. The method of claim 11, wherein the genetic chromosome doubling polypeptide allowing replication of the genome without cell division is selected from the group consisting of:
a) an amino acid sequence having at least 70% (e.g., at least 80%, 90%, 95%, or 98%) identical to SEQ ID NO: 24;
b) an amino acid sequence having at least 70% (e.g., at least 80%, 90%, 95%, or 98%) identical to SEQ ID NO: 25;
c) an amino acid sequence having at least 70% (e.g., at least 80%, 90%, 95%, or 98%) identical to SEQ ID NO: 26; or
d) an amino acid sequence having at least 70% (e.g., at least 80%, 90%, 95%, or 98%) identical to SEQ ID NO: 2.

13. The method of claim 11, wherein the genetic chromosome doubling polypeptide destabilizing tubulin polymerization is selected from the group consisting of:
a) an amino acid sequence having at least 70% (e.g., at least 80%, 90%, 95%, or 98%) identical to SEQ ID NO: 3; or
b) an amino acid sequence having at least 70% (e.g., at least 80%, 90%, 95%, or 98%) identical to SEQ ID NO: 28.

14. The method of claim 11, wherein the genetic chromosome doubling polypeptide altering cell cycle regulation is selected from the group consisting of:
a) an amino acid sequence having at least 70% (e.g., at least 80%, 90%, 95%, or 98%) identical to SEQ ID NO: 23;
b) an amino acid sequence having at least 70% (e.g., at least 80%, 90%, 95%, or 98%) identical to SEQ ID NO: 86;
c) an amino acid sequence having at least 70% (e.g., at least 80%, 90%, 95%, or 98%) identical to SEQ ID NO: 87;
d) an amino acid sequence having at least 70% (e.g., at least 80%, 90%, 95%, or 98%) identical to SEQ ID NO: 88;
e) an amino acid sequence having at least 70% (e.g., at least 80%, 90%, 95%, or 98%) identical to SEQ ID NO: 89;
f) an amino acid sequence having at least 70% (e.g., at least 80%, 90%, 95%, or 98%) identical to SEQ ID NO: 90;
g) an amino acid sequence having at least 70% (e.g., at least 80%, 90%, 95%, or 98%) identical to SEQ ID NO: 91;
h) an amino acid sequence having at least 70% (e.g., at least 80%, 90%, 95%, or 98%) identical to SEQ ID NO: 92;
i) an amino acid sequence having at least 70% (e.g., at least 80%, 90%, 95%, or 98%) identical to SEQ ID NO: 93;
j) an amino acid sequence having at least 70% (e.g., at least 80%, 90%, 95%, or 98%) identical to SEQ ID NO: 94;
k) an amino acid sequence having at least 70% (e.g., at least 80%, 90%, 95%, or 98%) identical to SEQ ID NO: 95;
l) an amino acid sequence having at least 70% (e.g., at least 80%, 90%, 95%, or 98%) identical to SEQ ID NO: 96;
m) an amino acid sequence having at least 70% (e.g., at least 80%, 90%, 95%, or 98%) identical to SEQ ID NO: 97;
n) an amino acid sequence having at least 70% (e.g., at least 80%, 90%, 95%, or 98%) identical to SEQ ID NO: 98;
o) an amino acid sequence having at least 70% (e.g., at least 80%, 90%, 95%, or 98%) identical to SEQ ID NO: 99;
p) an amino acid sequence having at least 70% (e.g., at least 80%, 90%, 95%, or 98%) identical to SEQ ID NO: 100;
q) an amino acid sequence having at least 70% (e.g., at least 80%, 90%, 95%, or 98%) identical to SEQ ID NO: 101;
r) an amino acid sequence having at least 70% (e.g., at least 80%, 90%, 95%, or 98%) identical to SEQ ID NO: 102; or
s) an amino acid sequence having at least 70% (e.g., at least 80%, 90%, 95%, or 98%) identical to SEQ ID NO: 103.

15. A method of producing a doubled haploid inducer (DHI) plant, the method comprising:
a) providing a transformable cell or tissue from a non-transgenic haploid inducer plant;
b) transforming the cell or tissue with a heterologous DNA construct comprising a genetic chromosome doubling agent; and
c) obtaining a double haploid inducer (DHI) line comprising the introduced genetic chromosome doubling agent.

16. The method of claim 15, wherein the non-transgenic haploid inducer comprises a loss-of-function mutation in a patatin-like phospholipase A2α gene.

17. The method of claim 16, wherein the loss-of-function mutation in the patatin-like phospholipase A2α gene is the MATRILINEAL (MATL) gene.

18. The method of claim 15, wherein the genetic chromosome doubling agent is selected from the group consisting of:
a) a polypeptide allowing replication of a genome without cell division;
b) a polypeptide destabilizing tubulin polymerization;
c) a polypeptide altering cell cycle regulation; and
d) combinations of the foregoing.

19. The method of claim 18, wherein the genetic chromosome doubling polypeptide allowing replication of the genome without cell division is selected from the group consisting of:
a) an amino acid sequence having at least 70% (e.g., at least 80%, 90%, 95%, or 98%) identical to SEQ ID NO: 24;
b) an amino acid sequence having at least 70% (e.g., at least 80%, 90%, 95%, or 98%) identical to SEQ ID NO: 25;
c) an amino acid sequence having at least 70% (e.g., at least 80%, 90%, 95%, or 98%) identical to SEQ ID NO: 26; or
d) an amino acid sequence having at least 70% (e.g., at least 80%, 90%, 95%, or 98%) identical to SEQ ID NO: 2.

20. The method of claim 18, wherein the genetic chromosome doubling polypeptide destabilizing tubulin polymerization is selected from the group consisting of:
a) an amino acid sequence having at least 70% (e.g., at least 80%, 90%, 95%, or 98%) identical to SEQ ID NO: 3; or
b) an amino acid sequence having at least 70% (e.g., at least 80%, 90%, 95%, or 98%) identical to SEQ ID NO: 28.

21. The method of claim 18, wherein the genetic chromosome doubling polypeptide altering cell cycle regulation is selected from the group consisting of:
a) an amino acid sequence having at least 70% (e.g., at least 80%, 90%, 95%, or 98%) identical to SEQ ID NO: 23;
b) an amino acid sequence having at least 70% (e.g., at least 80%, 90%, 95%, or 98%) identical to SEQ ID NO: 86;
c) an amino acid sequence having at least 70% (e.g., at least 80%, 90%, 95%, or 98%) identical to SEQ ID NO: 87;
d) an amino acid sequence having at least 70% (e.g., at least 80%, 90%, 95%, or 98%) identical to SEQ ID NO: 88;
e) an amino acid sequence having at least 70% (e.g., at least 80%, 90%, 95%, or 98%) identical to SEQ ID NO: 89;
f) an amino acid sequence having at least 70% (e.g., at least 80%, 90%, 95%, or 98%) identical to SEQ ID NO: 90;
g) an amino acid sequence having at least 70% (e.g., at least 80%, 90%, 95%, or 98%) identical to SEQ ID NO: 91;
h) an amino acid sequence having at least 70% (e.g., at least 80%, 90%, 95%, or 98%) identical to SEQ ID NO: 92;
i) an amino acid sequence having at least 70% (e.g., at least 80%, 90%, 95%, or 98%) identical to SEQ ID NO: 93;
j) an amino acid sequence having at least 70% (e.g., at least 80%, 90%, 95%, or 98%) identical to SEQ ID NO: 94;
k) an amino acid sequence having at least 70% (e.g., at least 80%, 90%, 95%, or 98%) identical to SEQ ID NO: 95;
l) an amino acid sequence having at least 70% (e.g., at least 80%, 90%, 95%, or 98%) identical to SEQ ID NO: 96;
m) an amino acid sequence having at least 70% (e.g., at least 80%, 90%, 95%, or 98%) identical to SEQ ID NO: 97;
n) an amino acid sequence having at least 70% (e.g., at least 80%, 90%, 95%, or 98%) identical to SEQ ID NO: 98;
o) an amino acid sequence having at least 70% (e.g., at least 80%, 90%, 95%, or 98%) identical to SEQ ID NO: 99;
p) an amino acid sequence having at least 70% (e.g., at least 80%, 90%, 95%, or 98%) identical to SEQ ID NO: 100;
q) an amino acid sequence having at least 70% (e.g., at least 80%, 90%, 95%, or 98%) identical to SEQ ID NO: 101;
r) an amino acid sequence having at least 70% (e.g., at least 80%, 90%, 95%, or 98%) identical to SEQ ID NO: 102; or
s) an amino acid sequence having at least 70% (e.g., at least 80%, 90%, 95%, or 98%) identical to SEQ ID NO: 103.

22. A method of producing a doubled haploid inducer (DHI) line, the method comprising:
a) providing a transformed first haploid inducer line comprising a heterologous DNA construct comprising a genetic chromosome doubling agent;
b) providing a second haploid inducer line containing a marker;
c) crossing the first haploid inducer line with the second haploid inducer line to produce a combination of maternal and diploid embryos;
d) selecting the maternal embryos based on the absence of the selectable marker from the second haploid inducer line; and
e) obtaining a double haploid inducer (DHI) line comprising the introduced genetic chromosome doubling agent, thereby producing the DHI line.

23. The method of claim 22, wherein the first haploid inducer comprises a loss-of-function mutation in a patatin-like phospholipase A2α gene.

24. The method of claim 23, wherein the loss-of-function mutation in the patatin-like phospholipase A2α gene is the MATRILINEAL (MATL) gene.

25. The method of claim 22, wherein the marker is selected from a selectable marker, a reporter gene, a visible endogenous morphological marker, and combinations thereof.

26. The method of claim 22 wherein the selectable marker is selected from the group consisting of GUS, PMI, PAT, and combinations thereof.

27. The method of claim 22, wherein the reporter gene is selected from the group consisting of GFP, RFP, CFP, and combinations thereof.

28. The method of claim 22, wherein the visible endogenous morphological marker is selected from the group consisting of B1, R-nj, R1-scm, anthocyanin pigments, and combinations thereof.

29. The method of claim 22, wherein the genetic chromosome doubling agent is a genetic chromosome doubling polypeptide.

30. The method of claim 29, wherein the genetic chromosome doubling polypeptide is selected from the group consisting of:
a) a polypeptide allowing replication of a genome without cell division;
b) a polypeptide destabilizing tubulin polymerization;
c) a polypeptide altering cell cycle regulation; and
d) combinations of the foregoing.

31. The method of claim 30, wherein the genetic chromosome doubling polypeptide allowing replication of the genome without cell division is selected from the group consisting of:
a) an amino acid sequence having at least 70% (e.g., at least 80%, 90%, 95%, or 98%) identical to SEQ ID NO: 24;
b) an amino acid sequence having at least 70% (e.g., at least 80%, 90%, 95%, or 98%) identical to SEQ ID NO: 25;
c) an amino acid sequence having at least 70% (e.g., at least 80%, 90%, 95%, or 98%) identical to SEQ ID NO: 26; or
d) an amino acid sequence having at least 70% (e.g., at least 80%, 90%, 95%, or 98%) identical to SEQ ID NO: 2.

32. The method of claim 30, wherein the genetic chromosome doubling polypeptide destabilizing tubulin polymerization is selected from the group consisting of:
a) an amino acid sequence having at least 70% (e.g., at least 80%, 90%, 95%, or 98%) identical to SEQ ID NO: 3; or
b) an amino acid sequence having at least 70% (e.g., at least 80%, 90%, 95%, or 98%) identical to SEQ ID NO: 28.

33. The method of claim 30, wherein the genetic chromosome doubling polypeptide altering cell cycle regulation is selected from the group consisting of:
a) an amino acid sequence having at least 70% (e.g., at least 80%, 90%, 95%, or 98%) identical to SEQ ID NO: 23;
b) an amino acid sequence having at least 70% (e.g., at least 80%, 90%, 95%, or 98%) identical to SEQ ID NO: 86;
c) an amino acid sequence having at least 70% (e.g., at least 80%, 90%, 95%, or 98%) identical to SEQ ID NO: 87;
d) an amino acid sequence having at least 70% (e.g., at least 80%, 90%, 95%, or 98%) identical to SEQ ID NO: 88;
e) an amino acid sequence having at least 70% (e.g., at least 80%, 90%, 95%, or 98%) identical to SEQ ID NO: 89;
f) an amino acid sequence having at least 70% (e.g., at least 80%, 90%, 95%, or 98%) identical to SEQ ID NO: 90;
g) an amino acid sequence having at least 70% (e.g., at least 80%, 90%, 95%, or 98%) identical to SEQ ID NO: 91;
h) an amino acid sequence having at least 70% (e.g., at least 80%, 90%, 95%, or 98%) identical to SEQ ID NO: 92;
i) an amino acid sequence having at least 70% (e.g., at least 80%, 90%, 95%, or 98%) identical to SEQ ID NO: 93;
j) an amino acid sequence having at least 70% (e.g., at least 80%, 90%, 95%, or 98%) identical to SEQ ID NO: 94;
k) an amino acid sequence having at least 70% (e.g., at least 80%, 90%, 95%, or 98%) identical to SEQ ID NO: 95;
l) an amino acid sequence having at least 70% (e.g., at least 80%, 90%, 95%, or 98%) identical to SEQ ID NO: 96;
m) an amino acid sequence having at least 70% (e.g., at least 80%, 90%, 95%, or 98%) identical to SEQ ID NO: 97;
n) an amino acid sequence having at least 70% (e.g., at least 80%, 90%, 95%, or 98%) identical to SEQ ID NO: 98;
o) an amino acid sequence having at least 70% (e.g., at least 80%, 90%, 95%, or 98%) identical to SEQ ID NO: 99;
p) an amino acid sequence having at least 70% (e.g., at least 80%, 90%, 95%, or 98%) identical to SEQ ID NO: 100;
q) an amino acid sequence having at least 70% (e.g., at least 80%, 90%, 95%, or 98%) identical to SEQ ID NO: 101;
r) an amino acid sequence having at least 70% (e.g., at least 80%, 90%, 95%, or 98%) identical to SEQ ID NO: 102; or
s) an amino acid sequence having at least 70% (e.g., at least 80%, 90%, 95%, or 98%) identical to SEQ ID NO: 103.

34. A method of eliminating or reducing the step of exogenously inducing chromosome doubling after embryo formation in the production of an inbred population, the method comprising crossing a doubled haploid inducer (DHI) line comprising an introduced genetic chromosome doubling agent with a second line and obtaining a doubled haploid progeny of the second line without a separate step of chromosome doubling due to exogenous treatment of a chromosome doubling agent, wherein the double haploid progeny does not comprise the introduced genetic chromosome doubling agent.

35. The method of claim 34, wherein the doubled haploid inducer comprises a loss-of-function mutation in a patatin-like phospholipase A2α gene.

36. The method of claim 35, wherein the loss-of-function mutation in the patatin-like phospholipase A2α gene is the MATRILINEAL (MATL) gene.

37. The method of claim 34, wherein the doubled haploid inducer (DHI) line expresses a marker gene.

38. The method of claim 37, wherein the marker gene is selected from a selectable marker, a reporter gene, a visible endogenous morphological marker, and combinations thereof.

39. The method of claim 38 wherein the selectable marker is selected from the group consisting of GUS, PMI, PAT, and combinations thereof.

40. The method of claim 38, wherein the reporter gene is selected from the group consisting of GFP, RFP, CFP, and combinations thereof.

41. The method of claim 38, wherein the visible endogenous morphological marker is selected from the group consisting of B1, R-nj, R1-scm, anthocyanin pigments, and combinations thereof.

42. The method of claim 34, wherein the introduced genetic chromosome doubling agent is a genetic chromosome doubling polypeptide.

43. The method of claim 42, wherein the genetic chromosome doubling polypeptide is selected from the group consisting of:
a) a polypeptide allowing replication of a genome without cell division;
b) a polypeptide destabilizing tubulin polymerization;
c) a polypeptide altering cell cycle regulation; and
d) combinations of the foregoing.

44. The method of claim 43, wherein the genetic chromosome doubling polypeptide allowing replication of the genome without cell division is selected from the group consisting of:
a) an amino acid sequence having at least 70% (e.g., at least 80%, 90%, 95%, or 98%) identical to SEQ ID NO: 24;
b) an amino acid sequence having at least 70% (e.g., at least 80%, 90%, 95%, or 98%) identical to SEQ ID NO: 25;
c) an amino acid sequence having at least 70% (e.g., at least 80%, 90%, 95%, or 98%) identical to SEQ ID NO: 26; or
d) an amino acid sequence having at least 70% (e.g., at least 80%, 90%, 95%, or 98%) identical to SEQ ID NO: 2.

45. The method of claim 43, wherein the genetic chromosome doubling polypeptide destabilizing tubulin polymerization is selected from the group consisting of:
a) an amino acid sequence having at least 70% (e.g., at least 80%, 90%, 95%, or 98%) identical to SEQ ID NO: 3; or
b) an amino acid sequence having at least 70% (e.g., at least 80%, 90%, 95%, or 98%) identical to SEQ ID NO: 28.

46. The method of claim 43, wherein the genetic chromosome doubling polypeptide altering cell cycle regulation is selected from the group consisting of:
a) an amino acid sequence having at least 70% (e.g., at least 80%, 90%, 95%, or 98%) identical to SEQ ID NO: 23;
b) an amino acid sequence having at least 70% (e.g., at least 80%, 90%, 95%, or 98%) identical to SEQ ID NO: 86;
c) an amino acid sequence having at least 70% (e.g., at least 80%, 90%, 95%, or 98%) identical to SEQ ID NO: 87;
d) an amino acid sequence having at least 70% (e.g., at least 80%, 90%, 95%, or 98%) identical to SEQ ID NO: 88;
e) an amino acid sequence having at least 70% (e.g., at least 80%, 90%, 95%, or 98%) identical to SEQ ID NO: 89;
f) an amino acid sequence having at least 70% (e.g., at least 80%, 90%, 95%, or 98%) identical to SEQ ID NO: 90;
g) an amino acid sequence having at least 70% (e.g., at least 80%, 90%, 95%, or 98%) identical to SEQ ID NO: 91;
h) an amino acid sequence having at least 70% (e.g., at least 80%, 90%, 95%, or 98%) identical to SEQ ID NO: 92;
i) an amino acid sequence having at least 70% (e.g., at least 80%, 90%, 95%, or 98%) identical to SEQ ID NO: 93;
j) an amino acid sequence having at least 70% (e.g., at least 80%, 90%, 95%, or 98%) identical to SEQ ID NO: 94;
k) an amino acid sequence having at least 70% (e.g., at least 80%, 90%, 95%, or 98%) identical to SEQ ID NO: 95;
l) an amino acid sequence having at least 70% (e.g., at least 80%, 90%, 95%, or 98%) identical to SEQ ID NO: 96;
m) an amino acid sequence having at least 70% (e.g., at least 80%, 90%, 95%, or 98%) identical to SEQ ID NO: 97;
n) an amino acid sequence having at least 70% (e.g., at least 80%, 90%, 95%, or 98%) identical to SEQ ID NO: 98;
o) an amino acid sequence having at least 70% (e.g., at least 80%, 90%, 95%, or 98%) identical to SEQ ID NO: 99;
p) an amino acid sequence having at least 70% (e.g., at least 80%, 90%, 95%, or 98%) identical to SEQ ID NO: 100;
q) an amino acid sequence having at least 70% (e.g., at least 80%, 90%, 95%, or 98%) identical to SEQ ID NO: 101;
r) an amino acid sequence having at least 70% (e.g., at least 80%, 90%, 95%, or 98%) identical to SEQ ID NO: 102; or
s) an amino acid sequence having at least 70% (e.g., at least 80%, 90%, 95%, or 98%) identical to SEQ ID NO: 103.

47. A method of maintaining a doubled haploid inducer (DHI) line comprising a genetic chromosome doubling agent, the method comprising:
a) providing a maintainer line comprising a heterologous maintainer construct, the maintainer construct comprising (i) a repressor or an inhibitor component for the genetic chromosome doubling agent, (ii) a pollen transmission prevention component, and (iii) a selectable marker component to identify seeds that contain the maintainer construct;
b) self-fertilizing the maintainer line having the maintainer construct and the genetic chromosome doubling agent;
c) identifying seeds containing the maintainer construct from the self-fertilization, wherein the maintainer construct is present in about 50% of the F1 seeds; and
d) growing the seeds containing the maintainer construct, thereby maintaining the DHI line in a diploid state for further seed increases or haploid induction crosses.

48. The method of claim 47, wherein the repressor or the inhibitor is a transcriptional repressor of the genetic chromosome doubling agent.

49. The method of claim 47, wherein the repressor or the inhibitor is an artificial micro RNA targeting the transcript of the genetic chromosome doubling agent.

50. The method of claim 47, wherein the selectable marker is a color-based marker suitable for seed sorting.

51. The method of claim 47, wherein the pollen transmission prevent component is a genetic element that reduces pollen viability thereby preventing the transmission of the maintainer construct through the pollen.

52. A doubled haploid inducer (DHI) maintainer line comprising a genetic chromosome doubling agent and a heterologous maintainer construct, the maintainer construct comprising (i) a repressor or an inhibitor component for the genetic chromosome doubling agent, (ii) a pollen transmission prevention component, and (iii) a selectable marker component to identify seeds that contain the maintainer construct.

53. The maintainer line of claim 52, wherein the repressor or the inhibitor is a transcriptional repressor of the genetic chromosome doubling agent.

54. The maintainer line of claim 52, wherein the repressor or the inhibitor is an artificial micro RNA targeting the transcript of the genetic chromosome doubling agent.

55. The maintainer line of claim 52, wherein the selectable marker is a color-based marker suitable for seed sorting.

56. The maintainer line of claim 52, wherein the pollen transmission prevent component is a genetic element that reduces pollen viability thereby preventing the transmission of the maintainer construct through the pollen.

57. The method of claim 1, wherein the first plant is a haploid inducer of a different plant species than the second plant.

58. The method of claim 1, wherein the first plant is used in a wide hybridization.

59. The method of claim 1, wherein the second plant species is corn, soybean, wheat, brassica, cotton or sorghum.

60. The method of claim 1, wherein the second plant species is a non-hybrid crop species.
